(19) United States
(12) Patent Application Publication
(10) Pub. No.: US 2023/0414147 A1
(43) Pub. Date: Dec. 28, 2023

(54) DEVICE FOR MEASURING BIOSIGNALS OF ELECTRICAL STIMULATION AND MANUFACTURING METHOD THEREOF

(71) Applicant: RESEARCH & BUSINESS FOUNDATION SUNGKYUNKWAN UNIVERSITY, Suwon-si (KR)

(72) Inventors: Donghee SON, Suwon-si (KR); Mikyung SHIN, Suwon-si (KR); Heewon CHOI, Suwon-si (KR); Yewon KIM, Suwon-si (KR); Sumin KIM, Suwon-si (KR)

(73) Assignee: RESEARCH & BUSINESS FOUNDATION SUNGKYUNKWAN UNIVERSITY, Suwon-si (KR)

(21) Appl. No.: 18/340,383

(22) Filed: Jun. 23, 2023

(30) Foreign Application Priority Data
Jun. 23, 2022 (KR) 10-2022-0076834

(51) Int. Cl. A61B 5/268; A61B 5/29; C08G 77/04; C08K 3/08; C09D 105/04; H01B 1/22; D01D 5/00; D06M 11/83; D06M 15/13

(52) U.S. Cl. CPC A61B 5/268; A61B 5/29; C08G 77/04; C08K 3/08; C09D 105/04; H01B 1/22; D06M 2101/30; D06M 11/83; D06M 15/13; A61B 2503/40; D10B 2509/00; D10B 2331/30; D01D 5/0038

(57) ABSTRACT

An embodiment of the present invention provides a device for measuring biosignals and electrical stimulation. The device for measuring biosignals and electrical stimulation includes a conductive composite including a self-healing polymer and liquid metal and exhibits low mechanical properties, excellent stress-relieving characteristics, and maintains conductivity.

### CROSS REFERENCE TO RELATED APPLICATION

The present application claims priority to Korean Patent Application No. 10-2022-0076834, filed Jun. 23, 2022, the entire contents of which is incorporated herein for all purposes by this reference.

### BACKGROUND OF THE INVENTION

### Field of the Invention

The present invention relates to a device for measuring biosignals and electrical stimulation, and more particularly, to a device for measuring electrical stimulation biosignals with low mechanical properties, excellent stress relief properties, and sustainable conductivity by incorporating a conductive composite including self-healing polymers and liquid metals.

### Description of the Related Art

In measuring biosignals and electrical stimulation, there is a demand for technologies related to flexible conductive materials.

Conventionally, a technology utilizing conductive materials including metals such as gold and silver has been used for measuring biosignals and electrical stimulation, but these conductive materials containing inorganic materials lose flexibility when the tissue is dynamic or deformed, leading to potential noise generation during biosignal measurement, which may result in the low signal-to-noise ratio, difficulties in accurate biosignal measurement, possible physical cracks in the conductive material itself, as well as physical cracks in the conductive materials themselves as well as in other structures in the device.

To address the issue of reduced flexibility in dynamic or deformable tissues, Thesis 1 (Sim, K. et al. An epicardial bioelectronic patch made from soft rubbery materials and capable of spatiotemporal mapping of electrophysiological activity. Nat. Electron. 3, 775-784 (2020)) introduced a technology in which an inherently flexible conductive composite material composed of one-dimensional silver (Ag) nanomaterials encapsulated in a gold (Au) shell and the rubbery polymer is applied to the epicardial mesh for a bidirectional electrical interface.

However, the technology introduced in thesis 1 has the drawback that fatigue caused by continuous cardiac contractions may lead to cracks in the polymer matrix. This issue may arise from the limited ability of the low-ductility polymer to efficiently release accumulated stresses within the rigid domains of the composite material.

Meanwhile, in the field of measuring biosignals and electrical stimulation, there is also a need for a technology allowing for uniform adhesion to tissue surfaces without the need for suturing.

However, conventional film-like polymers lack the necessary flexibility and conformability to wrap around the curved surfaces of tissues, making them unsuitable as films for implantable devices.

Additionally, conventional tissue adhesives require external factors such as temperature, ultraviolet (UV) light, or pressure, resulting in longer adhesion times and potential risks of tissue damage. Furthermore, in cases where tissues are not immobilized and exhibit non-periodic or periodic movement, the films or devices adhered to curved surfaces of the tissue may have a risk of detachment.

To address the mechanical adaptation and fixation issues related to dynamic tissues, thesis 2 (Lee, H. et al. Mussel-inspired surface chemistry for multifunctional coatings. Science 318, 426-430 (2007)) introduced an elastomer-based electronic device with an adhesive hydrogel layer incorporating polydopamine nanoparticles, inspired by mussels.

However, in thesis 1, improved adhesion strength and durability in dynamic tissues are required. Specifically, film-like elastomeric materials encounter challenges in conforming to the irregular curved surfaces of the epicardial tissue and maintaining integrity without cracks in films with nanometer thickness.

To address the above issues, the inventor of the present invention has developed a device for measuring biosignals and elastic electrical stimulation that exhibits high tissue adhesion strength and a conformable nature to cover curved surfaces comprehensively.

### SUMMARY OF THE INVENTION

The present invention has been conceived to solve the above problems, and it is an object of the present invention to provide a device for measuring biosignals and electrical stimulation with the inclusion of a network fiber layer including hydrogen bondable self-healing polymers and a hydrogel coating layer with tissue-adhesive hydrogel coating on the fiber layer, the hydrogel being characterized by infiltrating into the fiber layer.

The technical objects of the present invention are not limited to the aforesaid, and other objects not described herein with can be clearly understood by those skilled in the art from the descriptions below.

In order to accomplish the above objects, an embodiment of the present invention provides a device for measuring biosignals and electrical stimulation that incorporates conductive composite including self-healing polymers and liquid metal.

In an embodiment of the present invention, the liquid metal may be a eutectic alloy.

In an embodiment of the present invention, the liquid metal may include one or more selected from the group consisting of eutectic gallium-indium alloy, eutectic gallium-tin alloy, eutectic gallium-indium-tin alloy, and gallium.

In an embodiment of the present invention, the liquid metal may be 82 to 88 wt % in content relative to the total weight of the conductive composite, denoted as 100 wt %.

In an embodiment of the present invention, the liquid metal may be dispersed within the conductive composite.

In an embodiment of the present invention, the conductive composite may be disposed on a network fiber layer that includes fibers made of the self-healing polymer.

In an embodiment of the present invention, the conductive composite may be coupled to the network fiber layer.

In an embodiment of the present invention, the device may include a hydrogel coating layer coated on the network fiber layer and the conductive composite.

In an embodiment of the present invention, the hydrogel may be infiltrated into the fiber layer.

In an embodiment of the present invention, the network fiber layer may be porous.

In an embodiment of the present invention, the network fiber layer may include fibers with a diameter of 4.5 to 6.5 μm and pores with a diameter of 50 to 70 μm.

In an embodiment of the present invention, the hydrogel may penetrate less than 60% of the thickness of the network fiber layer from the contact surface with the network fiber layer.

In an embodiment of the present invention, the hydrogel may be a catechol-bonded polymer.

In an embodiment of the present invention, the hydrogel may undergo hydrogen bonding or hydrophobic interactions with tissues.

In an embodiment of the present invention, the self-healing polymer may include a polymer main chain, a first structural unit containing —HN—C(═O)—NH— capable of forming strong hydrogen bonds, and a second structural unit containing —HN—C(═O)—NH— capable of forming weak hydrogen bonds.

In an embodiment of the present invention, the polymer main chain may include at least one selected from polysiloxane and polydialkylsiloxane (where alkyl is C1 to C6), such as polydimethylsiloxane, polyethylene oxide (PEO), polypropylene oxide (PPO), polybutylene oxide (PBO), perfluoropolyether (PFPE), polyolefin, poly(ethylene-co-1-butylene), polybutadiene, hydrogenated polybutadiene, poly(ethylene oxide)-poly(propylene oxide) copolymer, poly(hydroxyalkanoate), styrene-butadiene copolymer (SB), styrene-butadiene-styrene copolymer (SBS), styrene-ethylene-butylene-styrene copolymer (SEBS), ethylene propylene diene rubber (EPDR), acrylic rubber, polychloroprene rubber, polyurethane, fluoro-rubber, butyl rubber, or silicone rubber.

In an embodiment of the present invention, the first structural unit may be represented by Formula 1:

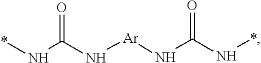

where Ar is a substituted or unsubstituted arylene group of C6 to C30 or a planar heteroarylene group of C3 to C30.

In an embodiment of the present invention, the second structural unit may be represented by Formula 2-1:

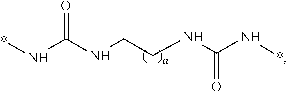

where a is an integer in the range of 5 to 20.

In an embodiment of the present invention, the second structural unit may be represented by Formula 2-2:

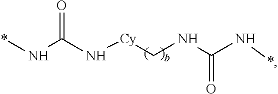

where Cy is a substituted or unsubstituted cyclic alkylene group of C5 to C30, and b is an integer of 1 or 3.

In an embodiment of the present invention, the biosignal may be a biosignal of human or animal tissue.

In an embodiment of the present invention, the biosignal may be a biosignal of the heart tissue in human or animal.

In order to accomplish the above object, an embodiment of the present invention provides a method for manufacturing an elastic device for measuring biosignals and electrical stimulation, the method including fabricating a network fiber layer by electrospinning self-healing polymer, arranging a conductive composite including a self-healing polymer and liquid metal onto the network fiber layer, and forming a hydrogel coating layer coated with hydrogel on the network fiber layer and the conductive composite.

In an embodiment of the present invention, the biosignal may be a biosignal of human or animal tissue.

In an embodiment of the present invention, the biosignal may be a biosignal of the heart tissue in human or animal.

In order to accomplish the above objects, an embodiment of the present invention provides a device for drug delivery or implantable in a body, the device including the device for measuring a biosignal of electrical stimulation, which is manufactured by the method for manufacturing a device for measuring biosignals and electrical stimulation.

### BRIEF DESCRIPTION OF THE DRAWINGS

FIG. 1 is a scanning electron microscope (SEM) image of the conductive composite (EGaIn/SHP) before (top) or after (bottom) stretching, according to an exemplary embodiment of the present invention.

FIG. 2 is a conceptual diagram illustrating stretching-induced cracks in a conductive composite using solid metal instead of liquid metal according to an embodiment of the present invention;

FIG. 3 is a conceptual diagram illustrating movement of charges from the conductive composite within a device for measuring biosignals and electrical stimulation, according to an embodiment of the present invention;

FIG. 4 is a conceptual diagram illustrating the principle of integration of a device for measuring biosignals and electric stimulation with tissue and the bonding mechanism between liquid metal and hydrogel according to an embodiment of the present invention. Specifically, it illustrates the bidirectional bonding principle between tissue and EGaIn through the Alg-CA coating layer, facilitated by metal-phenol coordination and ion interactions.

FIG. 5 is an SEM image of a network fiber layer according to an embodiment of the present invention;

FIG. 6 is a diagram illustrating an Alg-CA coating layer infiltrated into network fiber layer according to an embodiment of the present invention;

FIG. 7 is a conceptual diagram illustrating the infiltration of hydrogel into a network fiber layer and the principle of adhesion of the hydrogel to tissue according to an embodiment of the present invention;

FIG. 8 schematic diagram illustrating a self-assembly of PDMS-MPU-IU self-healing polymers;

FIG. 9 is a schematic diagram illustrating a chemical structure of network fiber layer;

FIG. 10 is a schematic diagram illustrating deformation of a network fiber layer under external stress;

FIG. 11 is a diagram illustrating an FEA model for comparing the distribution degrees of stress on the heart tissue after 15 minutes for E-SHN with Alg-CA and PDMS with Alg-CA;

FIG. 12 is a graph illustrating the adhesion strengths of a film with Alg-CA coating (black) and an EGaIn composite with Alg-CA coating (gray) to heart tissue according to an embodiment of the present invention;

FIG. 13 is a graph illustrating adhesive strengths of a solid film with Alg-CA (hatched bar) and E-SHN with Alg-CA (open bar);

FIG. 14 is a graph illustrating the adhesive retention forces of a film with Alg-CA (hatched bar) and E-SHN with Alg-CA (open bar) due to the substrate stress relief effect;

FIG. 15 is a graph illustrating periodic adhesion testing of Alg-CA-incorporated E-SHN under conditions simulating repetitive cardiac tissue movement;

FIG. 16 is photographs demonstrating adhesion of Alg-CA-infiltrated E-SHN to a rat heart within 0.5 seconds;

FIG. 17 is photographs demonstrating a wet adhesion test of Alg-CA-coated E-SHN on rat (top) and pig (bottom) tissues during PBS rinsing;

FIG. 18 is a diagram illustrating a device according to a comparative example and an device according to an embodiment of the present invention, both of which are adhered to tissues. Specifically, it shows the photograph curves comparing the tissue adhesion capability in 3-dimension (3D) between E-SHN (with a lower thickness of 100 μm) with no air gap and solid films with (with respective upper and middle thicknesses of 200 μm and 100 μm) with air gaps.

FIG. 19 is a graph illustrating a stress-strain relationship according to an embodiment of the present invention. Specifically, it illustrates the stress-strain relationship of the EGaIn/SHP composite (black) and the conventional solid filler (AgF) encapsulated SHP composite (hatched bar). As a control group sample, SHP standalone film (open bar) was used.

FIG. 20 is a graph illustrating residual strain (%) of a composite as a function of waiting time until recovery after one stretching-release cycle at 100% strain, according to an embodiment of the present invention;

FIG. 21 illustrates stress-strain curves (solid film —dash-single dotted lines, notched solid film—solid lines, E-SHN—dashed lines, and notched E-SHN—small single dotted lines) for evaluating destructiveness energy of each substrate material; The inserted graph (dashed rectangle) represents the elastic region of the curve for comparing the Young's modulus of the materials.

FIG. 22 is a graph illustrating fracture energies of a solid film and E-SHN;

FIG. 23 is a graph illustrating change in electrical resistance values as measured by mechanically stretching from 0% to the point of mechanical failure at a speed of 3 mm/min according to an embodiment of the present invention;

FIG. 24 is a graph illustrating resistance durability of EGaIn composite material during periodic testing of 0% or 50% tensile strain at a speed of 20 mm/min according to an embodiment of the present invention;

FIG. 25 is a graph illustrating impedance of a composite material as a function of strain at various frequencies (1, 10, 100, and 1,000 Hz) according to an embodiment of the present invention;

FIG. 26 is a graph illustrating a cyclic voltammetry of an EGaIn/SHP composite with (dashed line) and without (solid line) an Alg-CA coating layer according to an embodiment of the present invention;

FIG. 27 is photographs illustrating adhesive strengths of a substrate according to a comparative manufacturing example 1-1 and a conductive composite of according to embodiment 1 of the present invention;

FIG. 28 is a graph illustrating in vitro cell viabilities of an EGaIn composite on E-SHN with an Alg-CA coating layer (SAFIE device) according to an embodiment of the present invention;

Specifically, E-SHN and the EGaIn composites alone were used as control samples.

FIG. 29 is a graph illustrating the IR spectra of EGaIn with Alg-CA coating layer (dashed line) and only Alg-CA (solid line) according to an embodiment of the present invention;

FIG. 30 is a graph illustrating the high-resolution XPS spectra of C 1s for an EGaIn/SHP composite with an Alg-CA coating layer and only Alg-CA according to an embodiment of the present invention;

FIG. 31 is a graph illustrating the high-resolution XPS spectra of a Ga 3d and In 4d of the EGaIn/SHP composite with and without an Alg-CA coating layer according to an embodiment of the present invention;

FIG. 32 is a graph illustrating the high-resolution XPS spectra of In 3d for an EGaIn/SHP composite with and without an Alg-CA coating layer according to an embodiment of the present invention;

FIG. 33 is a graph illustrating the electrocardiograms (ECG) of rat heart stimulation using an EGaIn composite with Alg-CA (top) and without Alg-CA (bottom) according to an embodiment of the present invention;

Specifically, in the absence of Alg-CA, the EGaIn composite lost charge injection characteristics, and after several stimulations, the stimulation artifact (gray asterisk) and heart rhythm (arrow) were completely separated. The EGaIn composite with Alg-CA demonstrated stable myocardial capture (arrow) following the stimulation artifact.

FIG. 34 is photographs illustrating implantation processes of a SAFIE device on a rat heart (top images) and a surgical suturing of the SAFIE device without Alg-CA (bottom images) over time according to an embodiment of the present invention;

FIG. 35 is photographs illustrating the SAFIE device (top) and the device sutured onto the heart of a rat after three days of epicardial device implantation (bottom) according to an embodiment of the present invention; and

FIG. 36 is a photograph illustrating real-time ECG signals and bpm information received from four detection channels of the SAFIE device before and after drug injection in an induced heart disease model according to an embodiment of the present invention.

### DETAILED DESCRIPTION OF THE INVENTION

Hereinafter, the present invention will be described with reference to the accompanying drawings. However, the present invention may be embodied in many different forms and is not limited to the embodiments described herein. In order to clearly describe the present invention, parts irrelevant to the description may be omitted in the drawings, and similar reference numerals may be used for similar components throughout the specification.

Throughout the specification, when a part is said to be “connected (coupled, contacted, or combined)” with another part, this is not only “directly connected”, but also “indirectly connected” with another member in between. Also, when a part is said to “comprise” a certain component, this means that other components may be further included instead of excluding other components unless specifically stated otherwise.

The terminology used herein is for the purpose of describing particular embodiments only and is not intended to be limiting of the invention. As used herein, the singular forms are intended to include the plural forms as well, unless the context clearly indicates otherwise. It will be further understood that the terms “comprises” or “has,” when used in this specification, specify the presence of a stated feature, number, step, operation, component, element, or a combination thereof, but they do not preclude the presence or addition of one or more other features, numbers, steps, operations, components, elements, or combinations thereof.

Hereinafter, embodiments of the present invention will be described in detail with reference to the accompanying drawings.

In order to solve the above problems, an embodiment of the present invention provides a device for measuring biosignals and electrical stimulation that incorporates conductive composite including self-healing polymers and liquid metal.

FIG. 1 is a SEM image of the conductive composite (EGaIn/SHP) before (top) or after (bottom) stretching, according to an embodiment of the present invention.

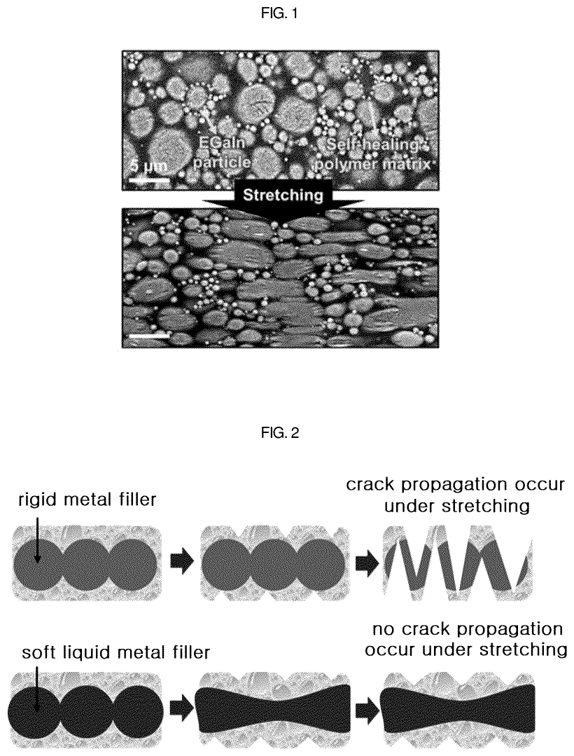

FIG. 2 is a conceptual diagram illustrating stretching-induced cracks in a conductive composite using solid metal instead of liquid metal according to an exemplary embodiment of the present invention.

With reference to FIGS. 1 and 2, the conductive composite incorporating self-healing polymers and liquid metal provides elasticity by including the self-healing polymers, facilitates easy bonding with a substrate containing the self-healing polymers, exhibits strong adhesion due to the self-healing properties, prevents detachment even during the movement of dynamic tissues, and effectively covers surfaces with curved tissue structures. Additionally, by including liquid metal, the conductive composite incorporating self-healing polymers and liquid metal prevents cracks from occurring under pressure or stretching in the device for measuring biosignals and electrical stimulation and allows for shape deformation to enable bonding with other liquid metals, thereby creating a pathway for the flow of charge.

In an embodiment of the present invention, the liquid metal may be an eutectic alloy. By including the eutectic alloy, the liquid metal within may remain in a liquid state within the body.

In an embodiment of the present invention, the liquid metal may include one or more selected from the group consisting of eutectic gallium-indium alloy, eutectic gallium-tin alloy, eutectic gallium-indium-tin alloy, and gallium. By including the metal, the conductive composite may be maintained as a liquid metal within the body, and the use of gallium-based eutectic alloys or gallium in the liquid metal is suitable for the electrodes and wiring of the biosignal measuring device due to their biocompatibility.

In an embodiment of the present invention, the liquid metal content may be 82 to 88 wt % relative to the total weight of the conductive composite, denoted as 100 wt %. Liquid metal content below 82 wt % may compromise the balance between elasticity and conductivity, while liquid metal content exceeding 88 wt % may interfere with the self-healing of the polymers through dynamic hydrogen bonding, potentially leading to liquid metal leakage.

In an embodiment of the present invention, the liquid metal may be dispersed within the conductive composite. By dispersing the liquid metal within the conductive composite, it is possible to effectively deliver electrical stimulation within the desired range of biological tissues and receive corresponding biological electrical signals.

In an embodiment of the present invention, the conductive composite may be positioned on a network fiber layer that includes fibers made of the self-healing polymer. The conductive composite attached onto the network fiber layer may be conformally mounted on curved surfaces of biological tissue.

In an embodiment of the present invention, the conductive composite may be coupled to the network fiber layer. The fibers and conductive composite share the same self-healing polymer composition, allowing fibers and conductive composite to bond together through mutual coupling when the conductive composite is arranged on the network fiber layer.

In an embodiment of the present invention, the device may include a hydrogel coating layer coated on the network fiber layer and the conductive composite.

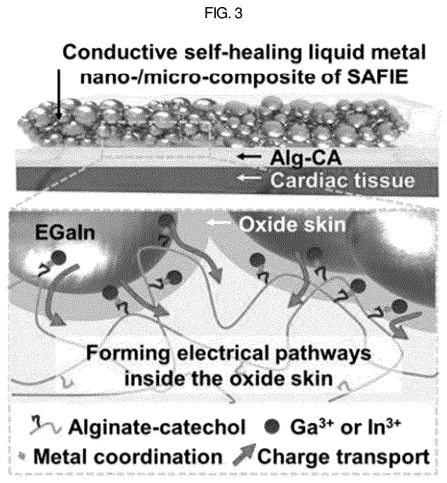

FIG. 3 is a conceptual diagram illustrating movement of charges from the conductive composite within a device for measuring biosignals and electrical stimulation, according to an exemplary embodiment of the present invention.

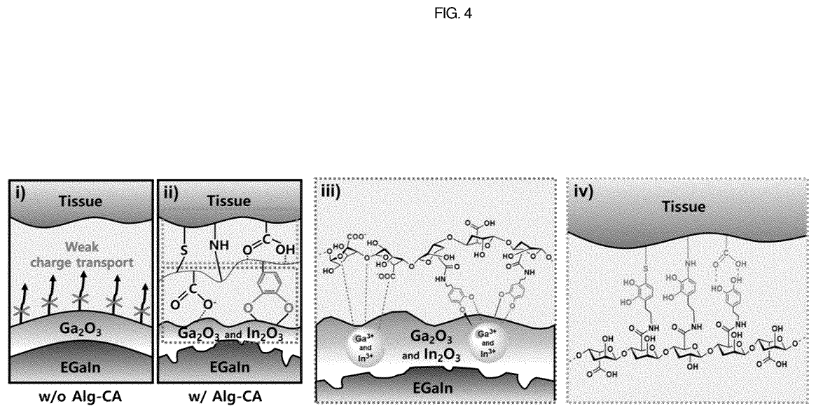

FIG. 4 is a conceptual diagram illustrating the principle of integration of a device for measuring biosignals and electric stimulation with tissue and the bonding mechanism between liquid metal and hydrogel according to an embodiment of the present invention. Specifically, it illustrates the bidirectional bonding principle between tissue and EGaIn through the Alg-CA coating layer, facilitated by metal-phenol coordination and ion interactions.

With reference to FIGS. 3 and 4, the hydrogel coating layer may bond to the conductive composite through coordination bonding with the metal cations present in the liquid metal included in the conductive composite, allowing for interaction with biological tissues through hydrogen bonding or hydrophobic interactions, and these bonds may serve as pathways for the flow of charge, facilitating the transmission of electrical stimulation.

In an embodiment of the present invention, the hydrogel may be infiltrated into the fiber layer.

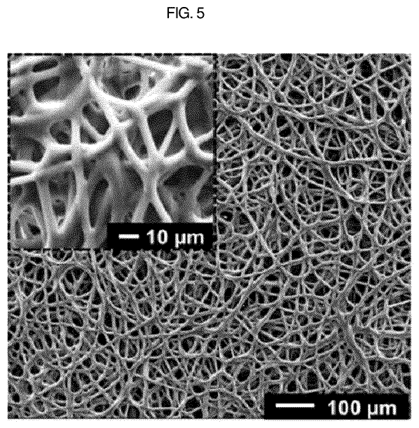

FIG. 5 is an SEM image of a network fiber layer according to an embodiment of the present invention.

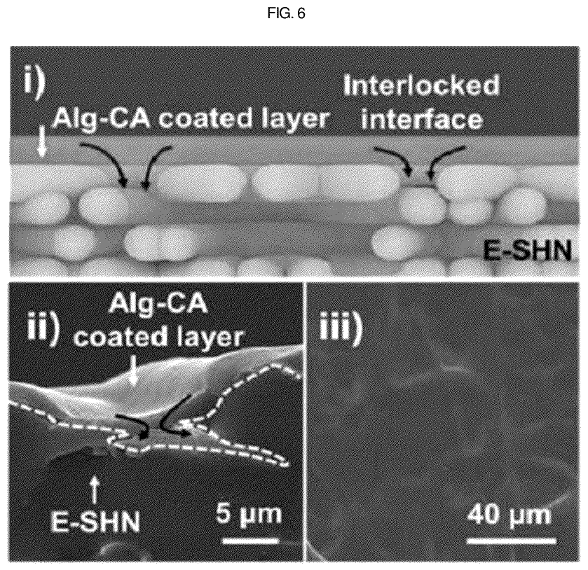

FIG. 6 is a diagram illustrating a fiber layer infiltrated into another fiber layer according to an embodiment of the present invention. In detail, the diagrams illustrate the morphological analysis of a device according to an embodiment of the present invention, which includes a network fiber layer equipped with hydrogel infiltration (E-SHN infiltrated with Alg-CA), and a device without hydrogel infiltration (solid film without Alg-CA). Specifically, (i) is an overall schematic view showing the coating layer of each device, (ii) is an SEM image of the cross-section of each device, and (iii) is a SEM image of the material's planar view.

With reference to FIGS. 5 and 6, the hydrogel, by infiltrating the fiber layer, efficiently undergoes elongation or deformation due to its structure physically entangled with the hydrogel, allowing for dissipating, dispersing, or absorbing energy exerted from the outside, and the elasticity of the fiber layer allows it to maintain adhesion even in dynamic tissues.

The physically entangled structure, formed by the hydrogel wrapping around the fibers and interacting either through intermolecular interactions within the hydrogel or with the fibers, may provide a physically bonded structure with the fibers serving as a supporting framework.

Preferably, the hydrogel infiltrates the fiber layer, forming a physically entangled structure with a portion of the fiber layer, which desirable as it may help maintain the inherent elasticity of the fiber layer while providing strong tissue adhesion. More preferably, the hydrogel may be penetrated less than 60% of the thickness of the network fiber layer from the contact surface with the network fiber layer. When the hydrogel penetrates more than 60% of the network fiber layer thickness, it may be inefficient in terms of cost and slightly reduce the flexibility of the device itself.

Moreover, the device for measuring biosignals and electrical stimulation possesses viscosity and flexibility, allowing the device to relieve stress from external forces and adapt to the shape of curved human or animal tissues, ensuring a close contact without any gaps between the device and the tissue surface.

In an embodiment of the present invention, the network fiber layer may be porous. The porous network fiber layer may be manufactured through electrospinning, and the porous structure allows for easy penetration of the hydrogel. When a device containing a non-porous fiber layer is used to adhere to tissues, the device may detach from the tissue over time.

In an embodiment of the present invention, the network fiber layer may include fibers with a diameter of 4.5 to 6.5 μm and pores with a diameter of 50 to 70 μm. A fiber diameter smaller than 4.5 μm may result in weak strength and potential breakage, and a fiber diameter larger than 6.5 μm may lead to reduced elasticity.

In an embodiment of the present invention, the hydrogel may be a catechol-bonded polymer.

In an embodiment of the present invention, the hydrogel may exhibit hydrogen bonding or hydrophobic interactions with tissues. The hydrogel may adhere quickly to the tissue due to hydrogen bonding or hydrophobic interactions, and intermolecular hydrogen bonding within the hydrogel may provide elasticity. The hydrophobic interactions may involve interactions between the hydrophobic ring structure of catechol groups and hydrophobic amino acids in the tissue. The main chain of the hydrogel may be alginic acid, and the functional group may include a catechol derivative including polyphenols containing pyrogallol moiety.

In an embodiment of the present invention, the self-healing polymer may include a polymer main chain, a first structural unit containing —HN—C(═O)—NH— capable of forming strong hydrogen bonds, and a second structural unit containing —HN—C(═O)—NH— capable of forming weak hydrogen bonds.

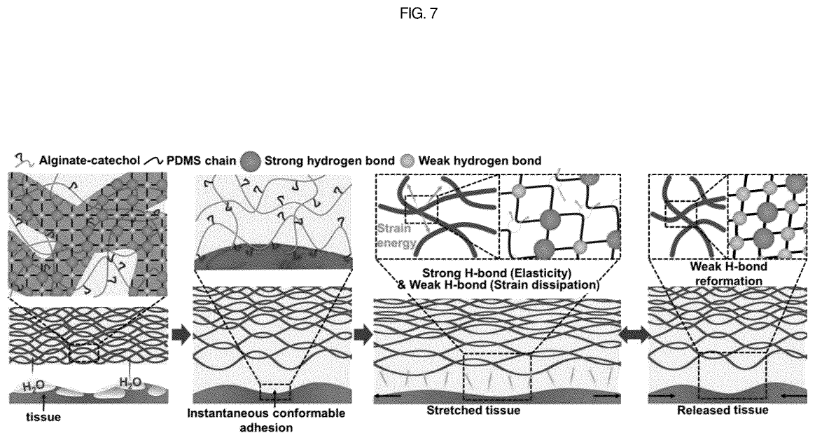

FIG. 7 is a conceptual diagram illustrating the infiltration of hydrogel into a network fiber layer and the principle of adhesion of the hydrogel to tissue according to an embodiment of the present invention.

With reference to FIG. 7, the structural entanglement and stress-relieving characteristics of the fiber layer may help disperse forces when the device is subjected to ongoing deformation even after attachment to tissue, thereby maintaining stable adhesion. In detail, when the weak hydrogen bonding region is subjected to tensile energy from the external environment, the intermolecular distance within this region increases compared to its state without tensile energy, but the intermolecular distance may decrease again once the external tensile energy disappears, due to the hydrogen bonding force in the weak hydrogen bonding region.

The polymer main chain of the self-healing polymer may be derived from a homopolymer, copolymer, or terpolymer that possesses flexibility, and the polymer may also be a crosslinked copolymer, block copolymer, or random copolymer, among others, without being particularly limited. For example, the polymer main chain may include at least one selected from polysiloxane and polydialkylsiloxane (where alkyl is C1 to C6), such as polydimethylsiloxane, polyethylene oxide (PEO), polypropylene oxide (PPO), polybutylene oxide (PBO), perfluoropolyether (PFPE), polyolefin, poly(ethylene-co-1-butylene), polybutadiene, hydrogenated polybutadiene, poly(ethylene oxide)-poly(propylene oxide) copolymer, poly(hydroxyalkanoate), styrene-butadiene copolymer (SB), styrene-butadiene-styrene copolymer (SBS), styrene-ethylene-butylene-styrene copolymer (SEBS), ethylene propylene diene rubber (EPDR), acrylic rubber, polychloroprene rubber, polyurethane, fluoro-rubber, butyl rubber, or silicone rubber. The polyolefin may be selected from polyethylene (PE), polypropylene (PP), polybutylene (PB), copolymers thereof, and mixtures thereof. The poly(ethylene oxide)-poly(propylene oxide) copolymer may be a block copolymer or a random copolymer.

The self-healing polymer includes a first structural unit containing —HN—C(═O)—NH— moiety capable of forming strong hydrogen bonds connected to the polymer main chain and a second structural unit containing —HN—C(═O)—NH— moiety capable of forming weak hydrogen bonds. The first structural unit capable of forming strong hydrogen bonds may form hydrogen bonds with all carbonyl groups of —HN—C(═O)—NH— moieties in one chain with all amino groups of —HN—C(═O)—NH— moieties in another chain. Additionally, the second structural unit capable of forming weak hydrogen bonds may include at least one portion that cannot form hydrogen bonds with carbonyl groups of —HN—C(═O)—NH— moieties in another chain while having amino groups of —HN—C(═O)—NH— moieties in one chain.

The strong hydrogen bonds provide elasticity and mechanical strength, while the weak hydrogen bonds contribute to extensibility and energy dissipation performance.

The first structural unit includes an aromatic ring and may be represented by the following Formula 1.

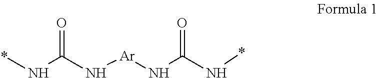

In Formula 1, —Ar— represents a substituted or unsubstituted arylene group of C6 to C30 or a planar heteroarylene group of C3 to C30.

The arylene group may be a single aromatic ring, a condensed ring formed by the fusion of two or more aromatic rings, or a combination of two or more aromatic rings connected by functional groups selected from single bonds, fluorenyl groups, cycloalkylene groups with a carbon chain length between C1 and C10, —O—, —S—, —C(═O)—, —CH(OH)—, —S(═O)2-, —Si(CH3)2-, —(CH2)p- (where 1≤p≤10), —(CF2)q- (where 1≤q≤10), —C(CH3)2-, —C(CF3)2-, —C(═O)NH—, and combinations thereof.

In Formula 1, —Ar— may include an aromatic group selected from substituted or unsubstituted phenylene, substituted or unsubstituted naphthylene, substituted or unsubstituted anthrylene, substituted or unsubstituted phenanthrylene, substituted or unsubstituted pyrenylene, substituted or unsubstituted peylenylene, substituted or unsubstituted fluorenylene, substituted or unsubstituted pentalene, substituted or unsubstituted pyrazole, substituted or unsubstituted imidazole, substituted or unsubstituted thiadiazole, substituted or unsubstituted triazole, substituted or unsubstituted carbazole, substituted or unsubstituted pyridine, substituted or unsubstituted pyridazine, substituted or unsubstituted pyrimidine, substituted or unsubstituted pyrazine, substituted or unsubstituted triazine, substituted or unsubstituted indazole, substituted or unsubstituted indoline, substituted or unsubstituted benzimidazole, substituted or unsubstituted benzothiazole, substituted or unsubstituted thienothiophene, substituted or unsubstituted benzothiophene, substituted or unsubstituted benzofuran, substituted or unsubstituted isoquinoline, and substituted or unsubstituted purine, or an aromatic group formed by connecting two or more of the aforementioned aromatic groups using a linker.

Here, the linker may be selected from a single bond, a substituted or unsubstituted alkylene group of C1 to C30, and a substituted or unsubstituted alkylene group of C1 to C30 including at least one link selected from —O—, —NR^{a}—, —C(═O)—, —OC(═O)—, —S(═O)₂—, —Si(R^{a}R^{b})₂—, and —C(═O)NR^{d}— (where R^{a}, R^{b}, R^{c}, and R^{d} are independently selected among hydrogen, alkyl groups of C1 to C10, alkenyl groups of C2 to C10, alkynyl groups of C2 to C10, aryl groups of C6 to C18, and heteroaryl groups of C2 to C18).

In Formula 1, —Ar— may be represented by the following Formula 1A or 1B.

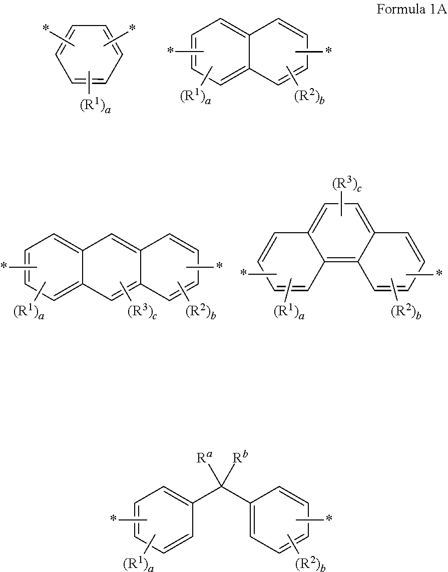

In Formula 1A, R¹ to R³ are independently hydrogen or alkyl groups of C1 to C6, and a, b, and c are integers representing the number of hydrogen atoms attached to the aromatic ring (e.g., they may be integers 0 to 4, 0 to 3, 0 to 2, or 1).

R^{a} and R^{b} are independently hydrogen, halogen, alkyl groups of C1 to C6, or fluoroalkyl groups of C1 to C6.

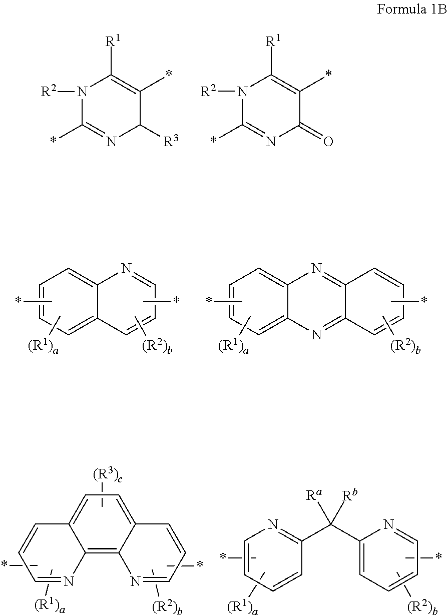

In Formula 1B, R¹ to R³ are independently hydrogen or alkyl groups of C1 to C6, and a, b, and c are integers representing the number of hydrogen atoms attached to the aromatic ring (e.g., they may be integers 0 to 4, 0 to 3, 0 to 2, or 1).

R^{a} and R^{b} are independently hydrogen, halogen, alkyl groups of C1 to C6, or fluoroalkyl groups of C1 to C6.

In Formulas 1A and 1B, the bond (indicated by *) connected to —HN—C(═O)—NH— may be arranged to have linearity, promoting the linear nature of the self-healing polymer. The aromatic rings of different chains in Formulas 1A and 1B may be oriented in a way that facilitates stacking and favorable hydrogen bonding.

The structural units that confer linearity to the self-healing polymer may be represented by Formula 1AA or 1BB.

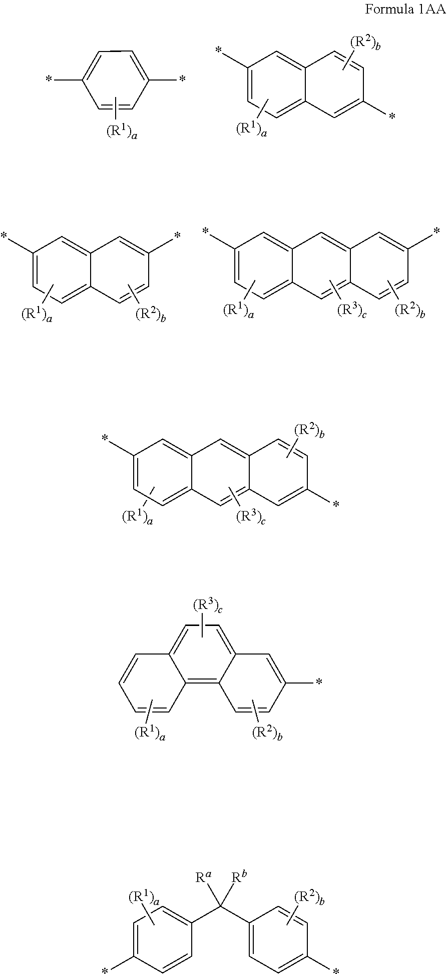

In Formula 1AA, R¹ to R³ are independently hydrogen or alkyl groups of C1 to C6, and a, b, and c are integers representing the number of hydrogen atoms attached to the aromatic ring (e.g., these may be integers 0 to 4, 0 to 3, 0 to 2, or 1), R^{a} and R^{b} are independently hydrogen, halogen alkyl groups of C1 to C6, or fluoroalkyl groups of C1 to C6.

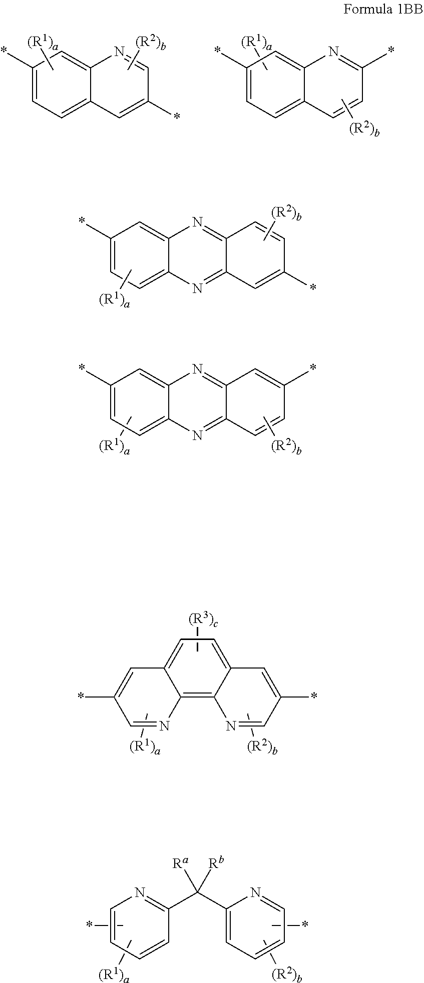

In Formula 1BB, R¹ to R³ are independently hydrogen or alkyl groups of C1 to C6, and a, b, and c are integers representing the number of hydrogen atoms attached to the aromatic ring (e.g., they may be integers 0 to 4, 0 to 3, 0 to 2, or 1), R^{a} and R^{b} are independently hydrogen, halogen alkyl groups of C1 to C6, or fluoroalkyl groups of C1 to C6.

The second structural unit includes an aliphatic chain or an aromatic (non-aromatic) ring system and may be represented by Formula 2-1 or 2-2.

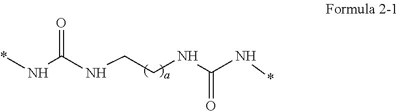

In Formula 2-1, the integer‘a’ is in the range of 5 to 20.

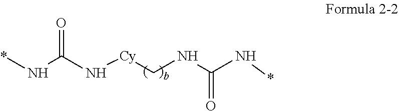

In Formula 2-2, ‘Cy’ represents a substituted or unsubstituted cyclic alkylene group of C5 to C30, and ‘b’ is an integer of 1 or 3. ‘Cy’ in Formula 2-2 may be represented by Formula 2-2A, 2-2B, or 2-2C.

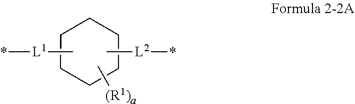

In Formula 2-2A, R¹ may be hydrogen or an alkyl group of C1 to C6, a represents the number of hydrogen atoms attached to the cyclohexyl ring, and L¹ and L² are independently a single bond or a substituted or unsubstituted alkylene group of C1 to C10.

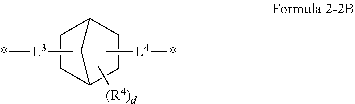

In Formula 2-2B, R² may be hydrogen or an alkyl group of C1 to C6, b represents the number of hydrogen atoms attached to the cyclohexyl ring, and L³ and L⁴ are independently a single bond or a substituted or unsubstituted alkylene group of C1 to C10.

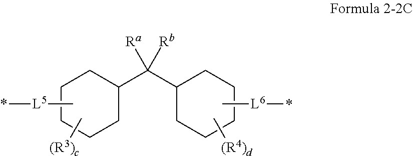

In Formula 2-2C, R³ and R⁴ are independently hydrogen or alkyl groups of C1 to C6, c and d represent the number of hydrogen atoms attached to the cyclohexyl ring, R^{a} and R^{b} are independently hydrogen, halogen, alkyl groups of C1 to C6, or fluoroalkyl groups of C1 to C6, and L⁵ and L⁶ are independently a single bond or a substituted or unsubstituted alkylene group of C1 to C10.

As a specific example of Formula 2-2A, it is possible to consider the Formula 2-2AA, as a specific example of Formula 2-2B, it is possible to consider the Formula 2-2BB, and as a specific example of Formula 2-2C, it is possible to consider the Formula 2-2CC.

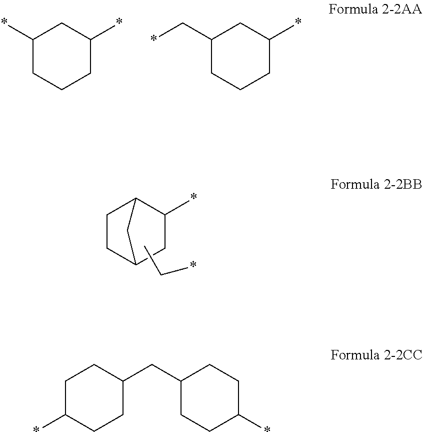

In Formula 2-2AA, 2-2BB, and 2-2CC, the hydrogen atoms present in each ring may be substituted with alkyl groups of C1 to C6.

When the structural unit is a moiety derived from 4,4′-methylenebis(phenylurea) (4,4′-first polydialkylsiloxane, and the polymer main chain is methylenebis(phenyl urea), MPU), and the second structural unit is a moiety derived from isophorone bisurea (IU), the self-healing polymer may include the repeating units of Formulas 3-1 and 3-2.

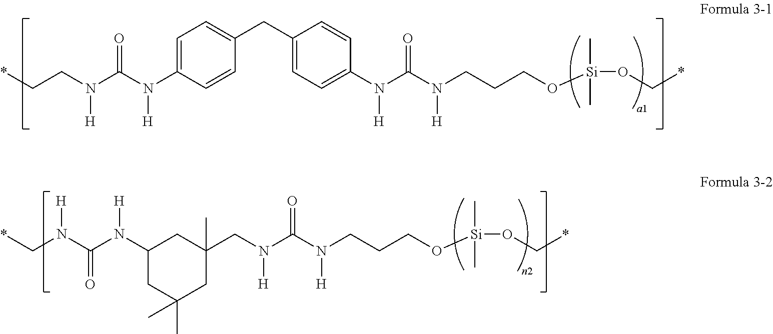

Here, n-1 in Formula 3-1 and in n-2 in Formula 3-2 are independently integers ranging from 10 to 200, e.g., from 20 to 150, 20 to 100, or 30 to 40.

In practical implementation, the relative mole ratio between the first structural unit, which provides strong hydrogen bonding, and the second structural unit, which provides weak hydrogen bonding, may be adjusted according to the desired properties of the composite. Increasing the content of the first structural unit enhances mechanical strength (Young's modulus and fracture energy), while increasing the content of the second structural unit improves self-healing rate and self-healing ability. For example, the first structural unit and the second structural unit may be included in the mole ratio of approximately 0.2:0.8 to 0.8:0.2, 0.3:0.7 to 0.7:0.3, 0.4:0.6 to 0.6:0.4, or 0.5:0.5.

The first structural unit and the second structural unit may be included in self-healing polymer in an amount of at least 0.01 mmol per mole of self-healing polymer, e.g., at least 0.02 mmol, at least 0.03 mmol, at least 0.04 mmol, or at least 0.05 mmol, and in an amount of up to 10 mmol, e.g., up to 9 mmol, up to 8 mmol, or up to 7 mmol, respectively. By controlling the composition within the aforementioned ranges, the elasticity, mechanical strength, and self-healing ability of the composite containing the self-healing polymer may be easily adjusted.

The self-healing polymer may have a number average molecular weight (Mn) in the range of approximately 10,000 or higher, e.g., around 11,000 or 12,000 or higher, and up to approximately 100,000 or lower, e.g., around 105,000, 110,000, or 115,000 or lower. When the number average molecular weight falls within the mentioned range, the self-healing polymer may exhibit excellent transparency and self-repairing characteristics. The number average molecular weight may be the polystyrene equivalent average molecular weight measured by gel permeation chromatography.

The polymer main chain may be derived from polymers with low glass transition temperatures. The glass transition temperature of the polymer may be around −40° C. or higher, e.g., around −30° C. or −20° C. or higher, and up to approximately 40° C. or lower, e.g., around 30° C. or 20° C. or lower. When the glass transition temperature falls within this range, the self-healing polymer may exhibit excellent transparency and self-restoration properties.

The multiple non-covalently hydrogen bonding interactions may lead to the crosslinking of the polymer main chains in the self-healing polymer, forming a supramolecular network (supramolecular elastomer network). That is, the chains of the self-healing polymer may self-assemble through continuous crosslinking via hydrogen bonding between —HN—C(═O)—NH— moieties. Such polymers crosslinked through hydrogen bonding exhibit faster self-healing rates and superior extensibility and self-recovery performance compared to structures crosslinked by covalent bonding. Accordingly, the composite material containing the self-healing polymer may exhibit high stretchability even under ambient conditions, allowing for rapid restoration of the network fiber layers incorporating the self-healing polymer during repetitive stretching, and it also possesses self-recovery characteristics, allowing for easy recombination and restoration of the original composite even upon damage. In one implementation, the composite material containing the self-healing polymer exhibits approximately 75% self-healing efficiency after 48 hours at room temperature (25° C.) and approximately 100% self-healing efficiency after 6 hours at 60° C.

The stretchability and self-recovery capabilities of the self-healing polymer are exemplified. The exemplified polymer has a polydimethylsiloxane (PDMS) backbone, with the first structural unit derived from 4,4′-methylenebis(phenyl urea) (MPU) and the second structural unit derived from isophorone bisurea (IU).

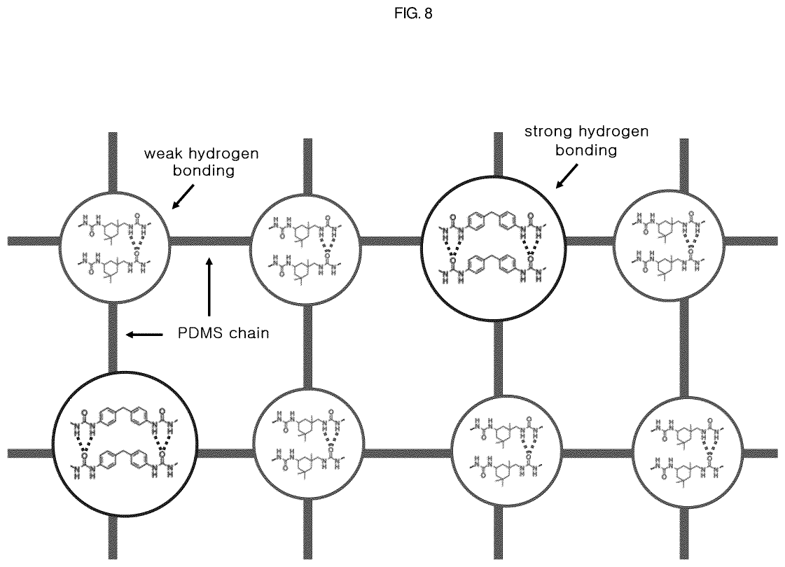

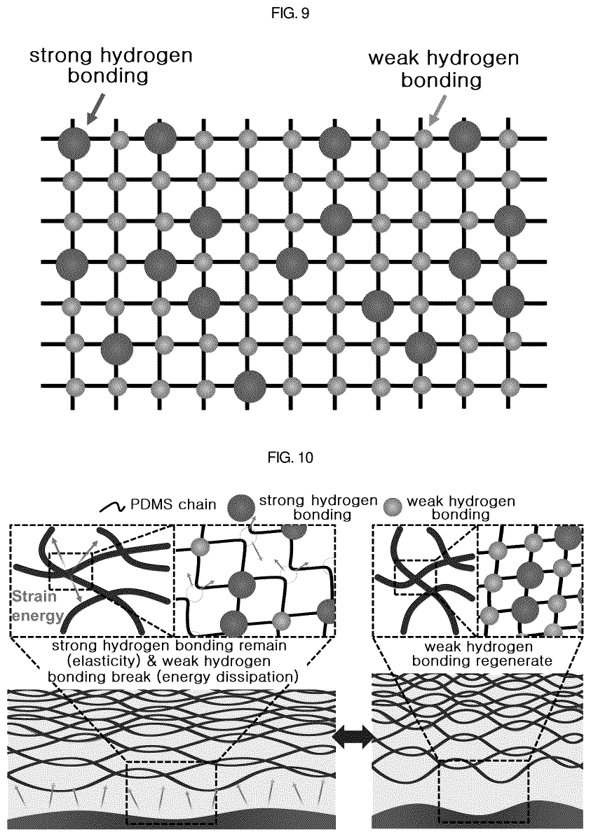

FIGS. 8 and 9 illustrate the self-assembly of the PDMS-MPU-IU self-healing polymer. FIG. 8 is a schematic diagram illustrating the self-assembly of the PDMS-MPU-IU self-healing polymer, and FIG. 9 is a schematic diagram illustrating the network fiber layer. With reference to FIG. 8, the hydrogen bonding between MPU units (larger circles) forms strong hydrogen bonds (cooperative H-bonding with four hydrogen bonds), while the hydrogen bonding between MPU-IU or IU-IU units (smaller circles) forms weak hydrogen bonds (anti-cooperative H-bonding with two hydrogen bonds). In a supramolecular network, strong hydrogen bonding forms strong crosslinking bonds, while weak hydrogen bonding forms weak crosslinking bonds.

When external stress is applied, the weak hydrogen bonding may preferentially break before the strong hydrogen bonding, allowing for stretchability and strain energy dissipation.

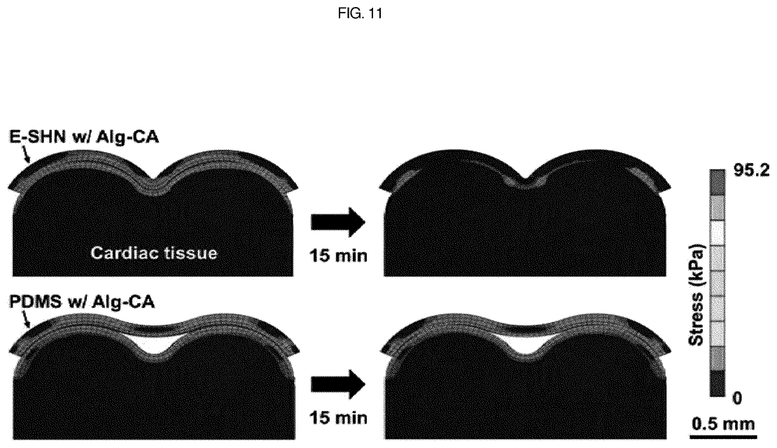

FIG. 10 illustrates the response of a composite material containing a supramolecular network of self-healing polymers to external stress. FIG. 10 is a schematic diagram illustrating deformation of a network fiber layer under external stress. With reference to FIG. 10, when the composite material (film) is stretched, it exhibits (i) high stretchability, ii) when a notched film (5 mm×5 mm) is stretched, strong hydrogen bonding prevents further crack propagation, while weak hydrogen bonding breaks, dissipating stress energy, and (iii) as weak hydrogen bonds break, the stress on strong hydrogen bonds decreases, and through hydrogen bond regeneration, self-recovery occurs.

The self-healing polymer forms a crosslinked matrix structure.

The tissue may be human or animal tissue, particularly rapidly pulsating cardiac tissue.

Hereinafter, a method for manufacturing an elastic device for measuring biosignals and electrical stimulation is described.

According to another embodiment of the present invention, a method for manufacturing an elastic device for measuring biosignals and electrical stimulation includes fabricating a network fiber layer by electrospinning self-healing polymer, arranging a conductive composite including a self-healing polymer and liquid metal onto the network fiber layer, and forming a hydrogel coating layer coated with hydrogel on the network fiber layer and the conductive composite.

Electrospinning the self-healing polymer creates pores in the fiber layer to fabricate a porous fiber layer, which allows for infiltration of hydrogel through these pores, leading to enhancement of the elasticity and tissue adhesion of the device used for measuring biosignals and electrical stimulation. In an embodiment of the present invention, the biosignals may be biosignals of human or animal tissues.

In an embodiment of the present invention, the biosignals may be biosignals of the heart tissue in human or animal.

Meanwhile, the device for measuring biosignals and electrical stimulation, manufactured by the method of the present invention, may be used as a device as an implantable device in the body.

Hereinafter, the above-described exemplary implementations are described in more detail through embodiments. However, the following embodiments are provided for illustrative purposes only and do not limit the scope of the invention.

### Synthesis Example 1: Synthesis of Self-Healing Polymer PDMS-MPU_{0.4}-IU_{0.5} (Hereinafter Referred to as SHP)

The solution of H2N-PDMS-NH2 (100 g, Mn=6000, 1 eq, Gelest Inc.) was prepared by dissolving it in anhydrous CHCl₃ (400 mL) and adding 10 mL of Et3N under argon at 0° C. After stirring for 1 hour, a mixture of 4,4′-methylenebis(phenyl isocyanate) (2.0 g, 0.4 eq.) and isophorone diisocyanate (2.7 g, 0.6 eq.) was added dropwise to the solution in CHCl₃. The mixture was stirred for 1 hour while maintaining a temperature of 0° C. using an ice bath. The resulting solution was kept at room temperature and stirred for 4 days. After the reaction, MeOH (15 mL) was added to remove the remaining isocyanate, and the mixture was stirred for 30 minutes. The obtained solution was concentrated to half of its volume, and 60 mL of MeOH was poured onto the precipitate. A white precipitate-like viscous liquid was formed, and the mixture was left undisturbed for 30 minutes. The clear solution on the top was decanted into another container, and 100 mL of CHCl₃ was added to dissolve the product. The solvent and a small amount of Et₃N were removed by vacuum evaporation after repeating the dissolution-precipitation-decantation process three times. The final product (PDMS-MPU0.4-IU0.6 self-healing polymer (referred to as SHP), was obtained with a yield of 65 g (63%). The molecular weight of the obtained self-healing polymer was as follows (measured by GPC, Mw=103,400; Mn=65,000 (D=1.6)).

¹H NMR (400 MHz, d5-THF): δ7.33 (d, J=8.0 Hz, 4H), 6.97 (d, J=8.0 Hz, 4H), 3.77 (s, 2H), 0.01 (b, 1325H). 13C NMR (400 MHz, CDCl₃): δ158.78, 139.18, 137.31, 125.34.

Material Acquisition

The sodium alginate (intermediate viscosity), dopamine hydrochloride, N-hydroxysuccinimide (NHS), 2-(N-morpholino)ethanesulfonic acid (MES solution), 1-dodecanethiol, and octadecyltrimethoxysilane (OTMS, 376213) were obtained from Sigma-Aldrich (USA). 1-Ethyl-3-(3-dimethylaminopropyl)carbodiimide hydrochloride (EDC) was obtained from Tokyo Chemical Inc. (Japan). PDMS (Sylgard 184) was obtained from The Dow Chemical Company (USA). EGaIn (eutectic gallium-indium alloy) was purchased from Alfa Aesar (USA). Ag flakes (AgFs, DSF-500MWZ-S) were purchased from Daejoo Electronics (South Korea). Chloroform was purchased from Samchun Chemical (South Korea).

### Manufacturing Example 1 Manufacturing of Conductive Composite (Hereinafter Referred to as EGaIn/SHP Nano-/Micro-Composite)

The liquid metal (eutectic gallium-indium, EGaIn) weighing 1 g was added to 5 mL of chloroform containing 0.5M 1-dodecanethiol, and the solution was uniformly dispersed using a tip sonicator (VCX750, SONICS & MATERIALS, INC., USA) with a power of 300 W for 5 minutes (10 seconds on, 5 seconds off pulses). After ultrasonic treatment, the solution was centrifuged at 4000 rpm for 5 minutes to remove the supernatant and precipitate the EGaIn. The precipitated EGaIn nano-/omicro-particles were washed several times with chloroform and then redispersed in the same solvent. Next, 1 mL of the redispersed EGaIn solution was mixed with 3 mL of chloroform containing dissolved SHP, and the mixture was stirred for 2 hours. All EGaIn and AgF composite solutions were cured at room temperature on OTMS-treated silicon wafers. After curing, a weak pressure was applied to induce conductivity pathways in the composite. Finally, the microstructure of the composite was observed using a field-emission scanning electron microscope (FE-SEM).

### Comparative Manufacturing Example 1-1

Except for using a PDMS solid film as the substrate, the manufacturing process followed the same method described in Manufacturing Example 1.

### Comparative Manufacturing Example 1-2

Except for dispersing AgF instead of EGaIn using a tip sonicator and omitting the process of separating and washing the solvent using a centrifuge, the manufacturing process followed the same method described in Manufacturing Example 1.

### Manufacturing Example 2 Manufacturing of Network Fiber Layer (Hereinafter Referred to as E-SHN)

The SHP (1 g) was dissolved in 30 mL of CHCl₃, and the SHP solution was prepared by stirring for 3 hours. Subsequently, the solution was drop-casted onto a Teflon dish with a diameter of 10 cm to produce a solid film. The network fiber layer (E-SHN) was fabricated by electrospinning the SHP solution.

Specifically, the SHP (1 g) was dissolved in 5 mL of CHCl₃ and stirred for 3 hours. The SHP solution (3 mL) was filled into a syringe equipped with a 21-gauge metal needle and connected to a syringe pump (New Era-300 Pump System, USA). Aluminum foil was applied to a grounded flat collector (NanoNC, South Korea). For electrospinning, the solution was ejected at a rate of 1.4 mL/h⁻¹ with a distance of 11 cm between the needle tip and the plate collector, at a voltage of 13 kV. Finally, 0.4 mL of the SHP solution was electrospun, and the resulting network fiber layer (E-SHN) had a thickness of 100 μm.

### Manufacturing Example 3 Manufacturing of Hydrogel (Hereinafter Referred to as Alq-CA)

Alg-CA was prepared through the coupling reaction of 1-ethyl-3-(3-dimethylaminopropyl)carbodiimide hydrochloride (EDC) and N-hydroxysuccinimide (NHS) at pH 4.5 to 4.8.

Specifically, alginate was dissolved in MES buffer (0.1M, adjusted to pH 4.5-4.8) to obtain a final concentration of 1% (w/v). The solution was then purged with nitrogen for 30 minutes. Dopamine hydrochloride, EDC, and NHS were added to the solution in an equimolar ratio with the carboxylic acid groups of the alginate backbone. The reaction was carried out at room temperature for 12 hours. To remove unreacted dopamine, the mixture was dialyzed in acidic distilled water (pH 5-5.5) for 12 hours and subsequently freeze-dried.

### Embodiment 1 Manufacturing of Device for Measuring Biosignals and Electrical Stimulation (Hereinafter Referred to as SAFIE)

To coat the Alg-CA onto the substrate where the conductive composite is attached to the E-SHN, SHP was dissolved in distilled water at a concentration of 3% by weight. The substrates, measuring 1.5×1.5 cm², were treated with oxygen plasma at 100 W for 6 minutes using a Femto Science plasma system (Korea). Immediately after plasma treatment, the Alg-CA solution was loaded onto the substrates and left at room temperature overnight for complete dehydration.

### Experimental Example 1

FE-SEM (JSM-7600F, JEOL) was used to observe the fine surface structure of the network fiber layer (E-SHN) and the EGaIn/SHP composite material and the cross-sectional structure of the network fiber layer (E-SHN) coated with Alg-CA. FIGS. 1, 5, and 6 are SEM images representing the cross-sections of EGaIn/SHP composite material, network fiber layer (E-SHN), and network fiber layer (E-SHN) coated with Alg-CA, respectively.

### Experimental Example 2

To theoretically analyze the stress-relieving effect of the viscoelastic material, finite element analysis (FEA) was conducted using commercial software (ANSYS, Ansys Inc., Canonsburg, PA, USA) assuming that PDMS polymer and E-SHN are subjected to repetitive pressure on heart tissue. The analysis results were presented in FIG. 11.

### Experimental Example 3: Extracorporeal Biological Tissue Adhesion Test

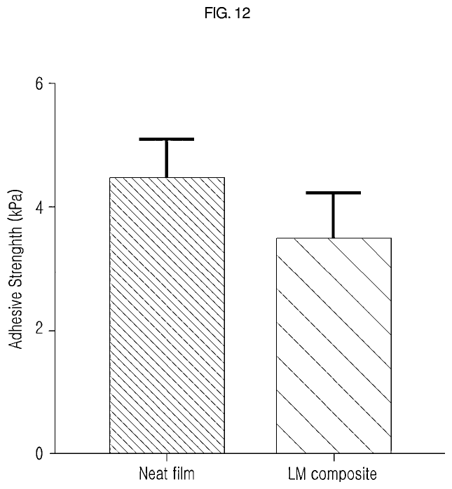

To investigate the tissue adhesion strength of the device for measuring biological signals of electrical stimulation of embodiment 1, porcine heart tissue (1×1 cm² or 1×2 cm²) was affixed to a polymeric substrate (e.g., polyethylene terephthalate (PET) film) using commercial super glue. Additionally, the E-SHN, EGaIn/SHP composite material and solid film (1×1 cm² or 1×2 cm²) attached to the substrate were compressed against the heart tissue. Shear stress or cyclic stress was applied to the terminals of the substrate using a 50N load cell at a speed of 10 mm/min⁻¹. Adhesion strength (kPa) and elongation (mm) were monitored using Instron Bluehill software. The test results were presented in FIGS. 12 to 15.

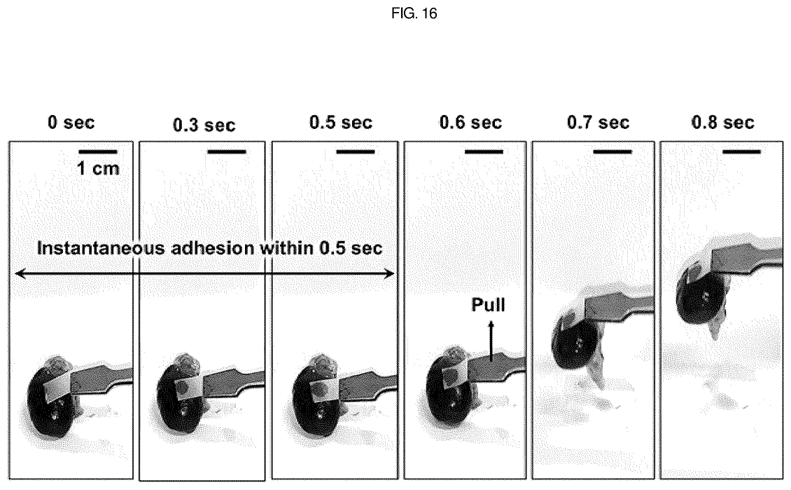

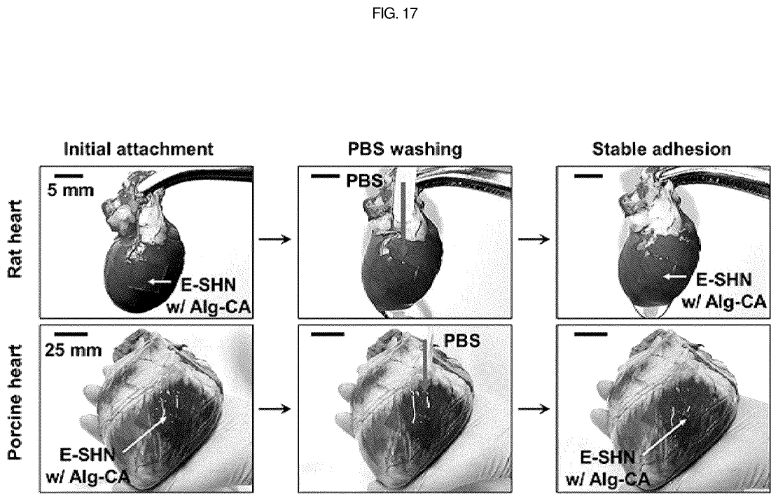

For qualitative adhesion testing, heart tissue was dissected from 11-week-old Sprague Dawley rats. Alg-CA (1×3 cm²) was swiftly attached to the heart tissue and immediately pulled upward to evaluate the tissue adhesion of SAFIE and E-SHN. The same adhesion test was performed after rinsing with phosphate-buffered saline (PBS, 1×, pH 7.4). The test results were presented in FIGS. 16 and 17.

### Experimental Example 4: Mechanical Characterization of Various Viscoelastic Material

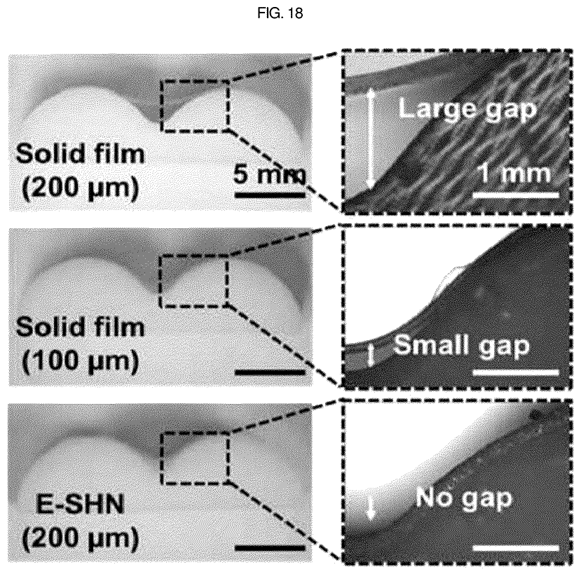

To measure the compatibility of E-SHN and solid films, models with E-SHN and solid film of thicknesses 100 μm or 200 μm were fabricated using a 3D printer and observed. FIG. 18 shows the images observed under an optical microscope.

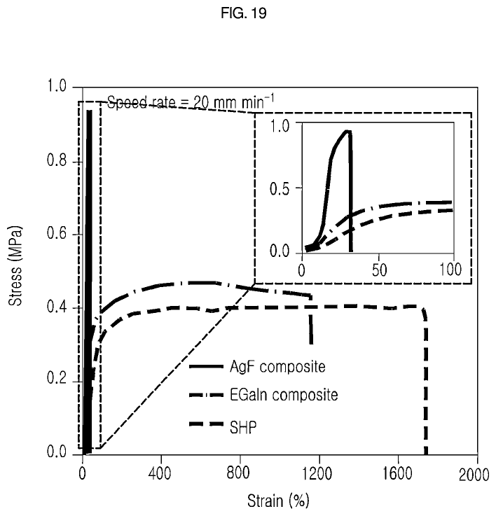

The tensile stress-strain curves of the solid film, E-SHN, and EGaIn/SHP composite material were recorded using a universal testing machine (UTM; Instron 34SC-1, USA). Samples with a width of 5 mm or 20 mm, thickness of 100 μm or 200 μm, and length of 10 mm were tested at a deformation rate of 20 mm/min⁻¹ (50N load cell). All experiments were performed four times to calculate the Young's modulus and residual strain of each sample. Furthermore, all experiments were performed with a lengthwise notch of 10 mm in the center of each sample to calculate the fracture strength of each sample. The results of the experiments were presented in FIGS. 19 to 22.

### Experimental Example 5: Measurement of Electrical and Mechanical Properties of EGaIn/SHP Nano/Micro Composite Materials

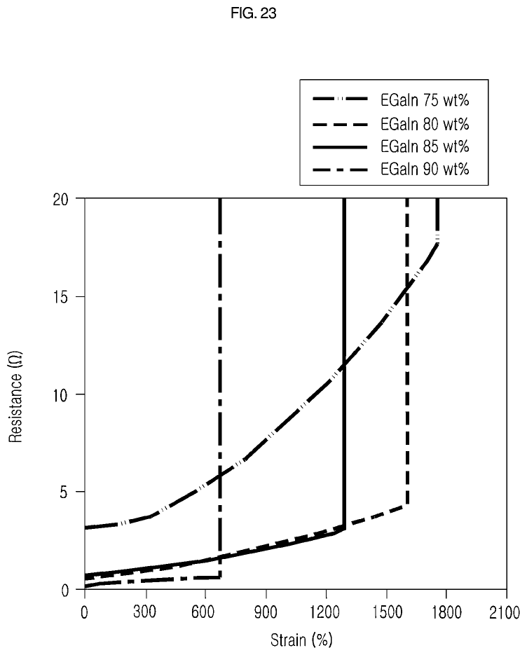

First, the electrical resistance of the stretched EGaIn/SHP nano/micro composite material was measured using a digital multimeter (Keithley 2450 source meter, Tektronics). The sample (initial length 3 mm, width 3 mm, thickness 150 μm) was fixed onto a motorized stage (SMC-100, Jeil Optical Systems) using double-sided tape and elongated at a speed of 3 mm/min⁻¹. The results of the experiment were presented in FIG. 23.

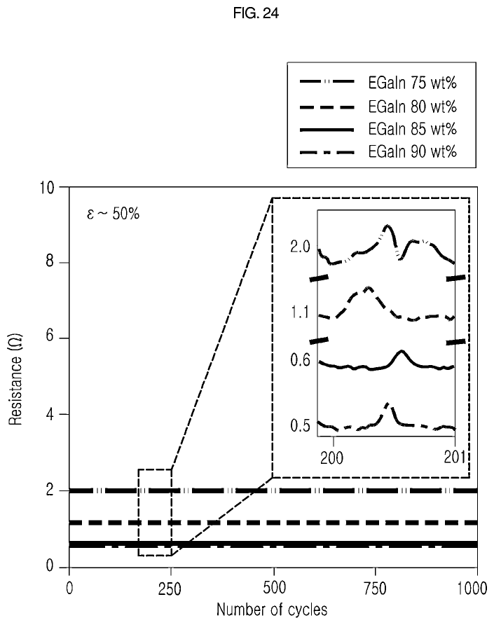

Second, for cyclic testing, the resistance was measured at 0% and 50% strain, and the samples were subjected to 1,000 cycles at a speed of 20 mm/min⁻¹. In all cases, EGaIn was used to form deformable electrical contacts between the composite and the multimeter wires. The results of the experiment were presented in FIG. 24.

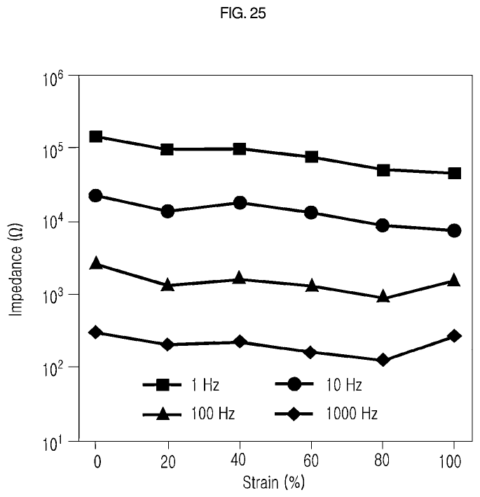

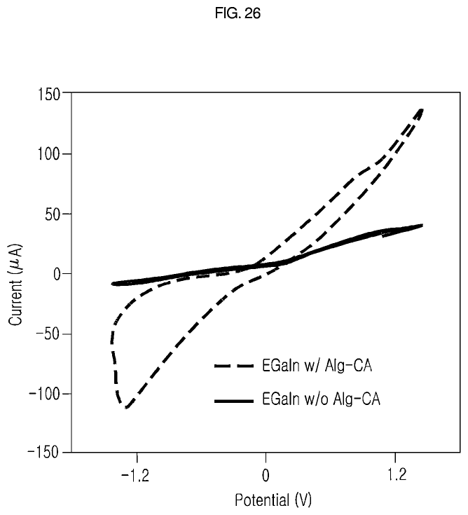

Third, the electrochemical impedance and cyclic voltammetry of the composite were monitored using a potentiostat (ZIVE sp1, ZIVE LAB) in the frequency range of 1 Hz to 1 kHz (potential range: −1.2 V to +1.2 V, scan rate: 50 mV/s⁻¹). The sample (0.5×0.5 cm² area) was immersed in PBS. The Ag/AgCl reference electrode and platinum counter electrode were used. The results of the experiment were presented in FIGS. 25 and 26.

### Experimental Example 6: Self-Adhesion Test Between EGaIn/SHP Composite and SHP Substrate

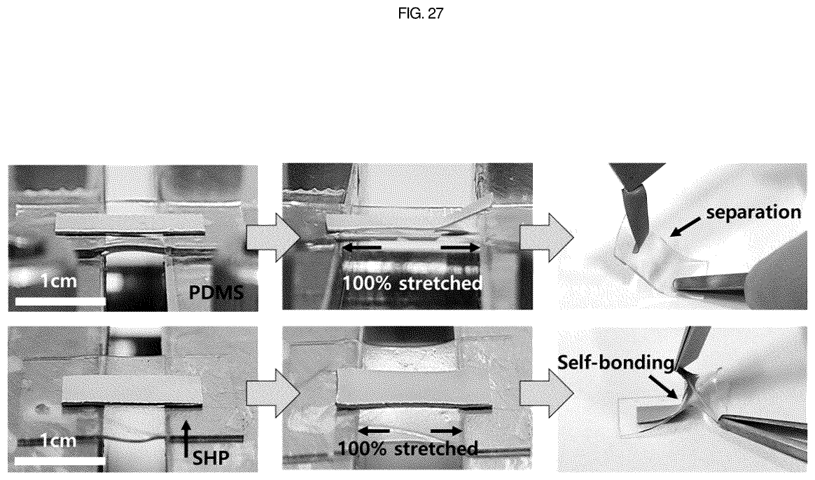

The EGaIn/SHP composite, cut into a size of 0.5×1.5 cm², was placed on SHP and PDMS substrates measuring 1×2.5 cm² and, after approximately 10 minutes, subjected to a 100% strain using a manual stretcher from an initial length of approximately 5 mm to observe whether separation occurred between the composite and the substrates. Furthermore, after removing the sample from the stretcher, the substrate and composite were pulled in opposite directions using tweezers to confirm whether separation occurred. The results of the experiment were presented in FIG. 27.

### Experimental Example 7: Assessment of Biocompatibility of SAFIE Device

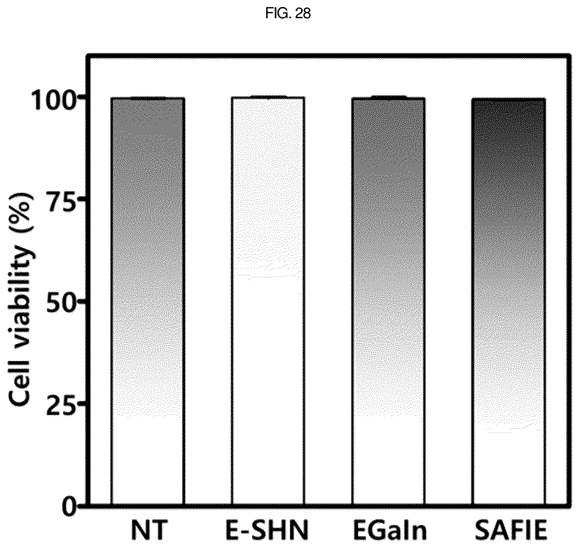

To assess the biocompatibility of the SAFIE device developed in this study, cell tests were conducted using mouse fibroblast cells (L929). Both E-SHN and EGaIn/SHP composite materials, the combined SAFIE device, were tested, and the number of living cells and dead cells were compared after a maximum of 72 hours to obtain the results. The results of the experiment were presented in FIG. 28.

### Experimental Example 8: Assessment of Chemical Bonding Changes of EGaIn/SHP Composite with and without Alg-CA Coating

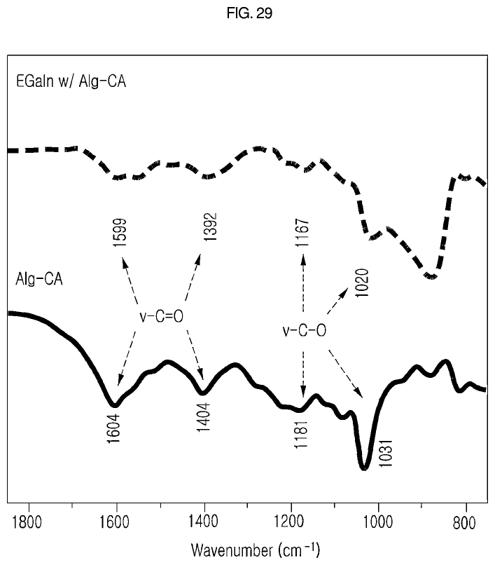

To conduct FTIR and XPS analysis, a sample containing only EGaIn particles, a sample with EGaIn-Alg-CA binding, and a sample containing only Alg-CA were prepared. First, the first sample was prepared by adding 36 mg of EGaIn to 3 ml of ultrapure water, and the second sample was prepared by adding 36 mg of EGaIn to 3 ml of 0.3 w/v % Alg-CA solution, and both samples were sonicated by a tip sonicator for 1 hour. The third sample was prepared using 0.3 w/v % Alg-CA solution. Each sample was dropped onto a 1×1 cm² glass substrate, dried completely, and then subjected to analysis using an X-ray Photoelectron Spectrometer (XPS, ESCALAB250, Thermo, USA) and Fourier-Transform Infrared Spectrometer (FTIR, IFS-66/S, TENSOR27, Bruker, USA). The results of the experiment were presented in FIGS. 29 to 31.

### Experimental Example 9: Assessment of In Vivo Operation of SAFIE Device

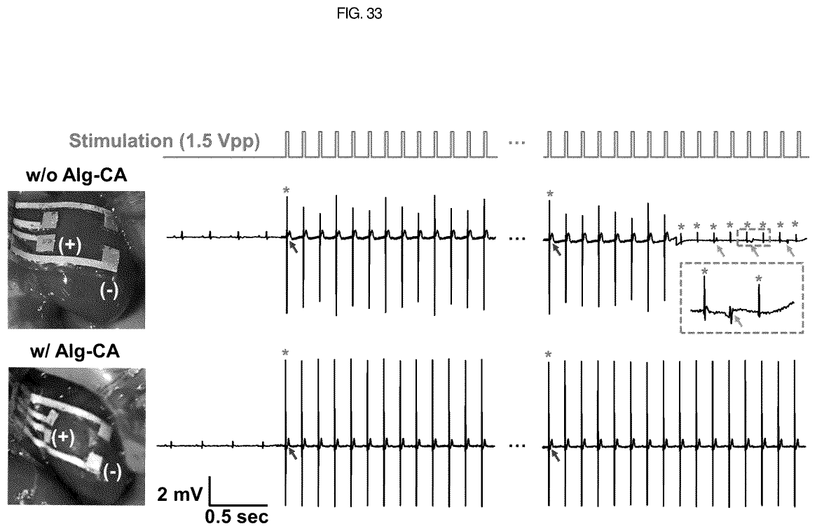

To confirm the proper functioning of the device in vivo, the device was attached to the hearts of actual 11-week-old Sprague Dawley rats, and electrocadiogram was measured using a data acquisition system (Powerlab 8/35, AD Instruments, Australia), applying square pulses of 1.5 Vpp, 1 ms pulse width, and 6 Hz frequency using a pulse stimulator (model 2100, AM systems). Additionally, the actual device was implanted in the rats for 3 days, and ECG measurements were taken without anesthesia to compare the effects of Alg-CA coating. The rats were intravenously injected with a drug (diltiazem), and the induced arrhythmias were measured. The results of the above experiments are presented in FIGS. 33 to 36.

Hereinafter, descriptions are made along with the graphs representing the results of the experimental examples.

FIG. 11 is a diagram illustrating an FEA model for comparing the distribution degree of stress on the heart tissue after 15 minutes for E-SHN with Alg-CA and PDMS with Alg-CA.

With reference to FIG. 11, it may be observed that after 15 minutes, E-SHN with Alg-CA has significantly reduced stress on the heart compared to PDMS with Alg-CA, indicating that the stress is almost eliminated.

FIG. 12 is a graph representing the adhesion strengths of a film with Alg-CA coating (black) and an EGaIn composite with Alg-CA coating (gray) to heart tissue according to an embodiment of the present invention.

With reference to FIG. 12, it may be observed that the adhesion strength of the EGaIn composite with Alg-CA coating is approximately 3-4 kPa.

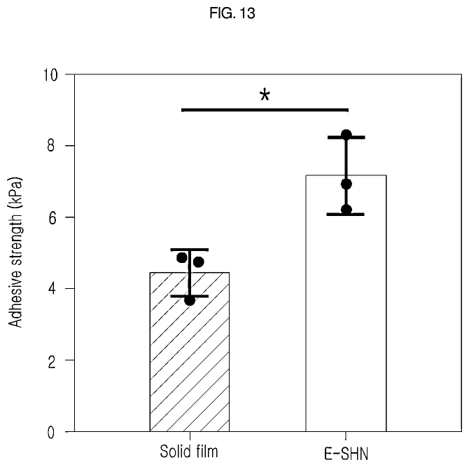

FIG. 13 is a graph illustrating adhesive strengths of a solid film with Alg-CA (dashed bar) and E-SHN with Alg-CA (open bar).

With reference to FIG. 13, it may be observed that the adhesion strength of E-SHN with Alg-CA is significantly higher than that of the solid film with Alg-CA, and this is because E-SHN exhibits better physical bonding with the Alg-CA layer.

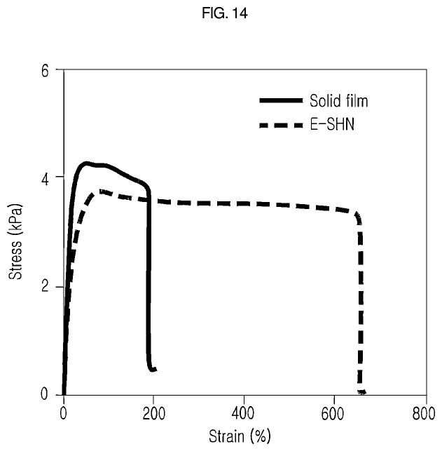

FIG. 14 is a graph illustrating the adhesive retention forces of a solid film with Alg-CA (solid line) and E-SHN with Alg-CA (dashed line) due to the substrate stress relief effect.

With reference to FIG. 14, it may be observed that the strain in response to stress is approximately three times higher in E-SHN compared to the film with Alg-CA.

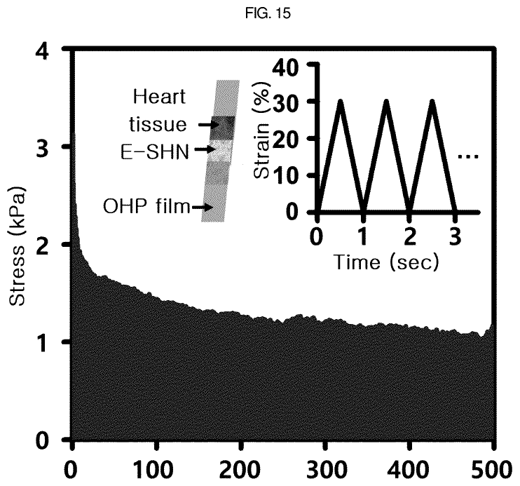

FIG. 15 is a graph illustrating periodic adhesion testing of Alg-CA-incorporated E-SHN under conditions simulating repetitive cardiac tissue movement.

With reference to FIG. 15, it is possible to observe the mechanical reliability of the adhesive performance of E-SHN with Alg-CA during 500 cycles of repetitive stretching and releasing. The inserted diagram indicates that each cycle lasts for 1 second and the stretching occurs at a strain of 30%.

FIG. 16 is photographs demonstrating adhesion of Alg-CA-infiltrated E-SHN to a rat heart within 0.5 seconds.

With reference to FIG. 16, it may be observed that the Alg-CA-infiltrated E-SHN adheres to the heart of a rat within 0.5 seconds.

FIG. 17 is photographs demonstrating a wet adhesion test of Alg-CA-infiltrated E-SHN on rat (top) and pig (bottom) tissues during PBS rinsing.

With reference to FIG. 17 it may be observed that the Alg-CA-infiltrated E-SHN maintains adhesion to both rat (top) and pig (bottom) tissues during PBS rinsing.

FIG. 18 is a photograph demonstrating a device according to a comparative example and an device according to an embodiment of the present invention, both of which are adhered to tissues. Specifically, it shows the photograph curves comparing the tissue adhesion capability in 3-dimension (3D) between E-SHN (with a lower thickness of 100 μm) with no air gap and solid films with (with respective upper and middle thicknesses of 200 μm and 100 μm) with air gaps.

With reference to FIG. 18, it may be observed that the solid film exhibits poor conformability, leading to significant gaps when placed on a three-dimensional structure, while E-SHN shows no gaps, indicating superior adhesion. It may be observed that even when the solid film is as thin as 100 μm, small gaps occur, whereas E-SHN does not exhibit any gaps even at a thicker thickness of 200 μm.

FIG. 19 is a graph illustrating a stress-strain relationship according to an embodiment of the present invention. Specifically, it illustrates the stress-strain relationship of the EGaIn/SHP composite (dash-single dotted line) and the conventional solid filer (AgF)/SHP composite (solid line). As a control group sample, SHP solid film (dashed line) was used.

With reference to FIG. 19, it may be observed that the GaIn/SHP composite exhibits a stress-strain relationship similar to that of the SHP film, while the AgF/SHP composite shows significantly higher modulus and stress values and lower strain. Therefore, the composite made with solid filler AgF exhibits increased stiffness and significantly reduced elongation, whereas the composite made with liquid filler EGaIn/SHP shows mechanical properties similar to the original polymer (SHP).

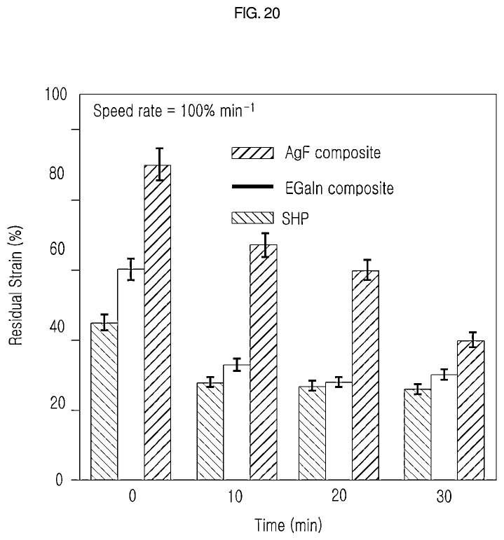

FIG. 20 is a graph illustrating residual strain (%) of a composite as a function of waiting time until recovery after one stretching-release cycle at 100% strain, according to an embodiment of the present invention.

With reference to FIG. 20, it may be observed that the AgF/SHP composite exhibits relatively higher strain, while the EGaIn/SHP composite shows strain similar to SHP, indicating that the EGaIn/SHP composite possesses properties similar to SHP compared to the AgF/SHP composite.

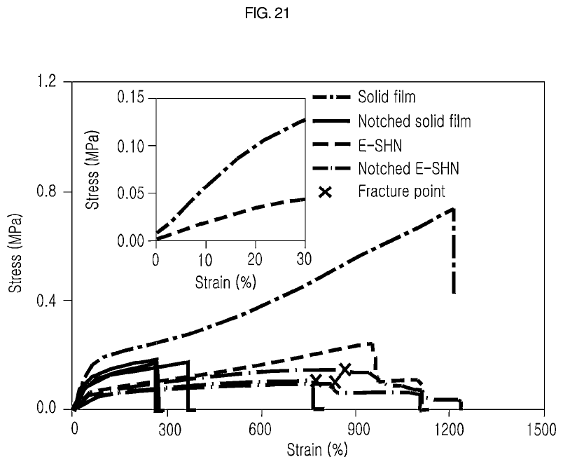

FIG. 21 illustrates stress-strain curves (solid film—dash-single dotted lines, notched solid film—solid lines, E-SHN—dashed lines, and notched E-SHN—small single dotted lines) for evaluating destructiveness energy of each substrate material. The inserted graph (dashed rectangle) represents the elastic region of the curve for comparing the Young's modulus of the materials.

With reference to FIG. 21, it may be observed that E-SHN has a smaller Young's modulus compared to the solid film, and when comparing notched samples, it exhibits significantly higher strain values, indicating superior stress-energy dissipation capability.

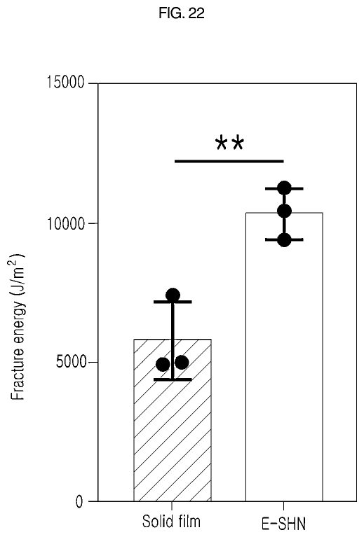

FIG. 22 is a graph illustrating fracture energies of a solid film and E-SHN.

With reference to FIG. 22, it may be observed that E-SHN has a significantly higher fracture energy compared to the solid film, indicating that E-SHN has a greater resistance to fracture.

FIG. 23 is a graph illustrating change in electrical resistance values as measured by mechanically stretching from 0% to the point of mechanical failure at a speed of 3 mm·min⁻¹ according to an embodiment of the present invention.

With reference to FIG. 23, it may be observed that the sample with 80 wt % EGaIn exhibited the highest elasticity, but the electrical resistance increased more rapidly with tensile strain compared to samples with 85 wt % EGaIn and 90 wt % EGaIn. In the case of 90 wt % EGaIn, it may be observed that the elasticity is lower compared to 80 wt % EGaIn and 85 wt % EGaIn.

FIG. 24 is a graph illustrating resistance durability of EGaIn composite material during periodic testing of 0% or 50% tensile strain at a speed of 20 mm·min⁻¹ according to an embodiment of the present invention.

With reference to FIG. 24, it may be observed that the EGaIn/SHP composite maintains stable electrical characteristics even under repeated deformation, as demonstrated by a cyclic test of 1000 repetitions at a constant speed and strain rate.

FIG. 25 is a graph illustrating impedance of a composite material as a function of strain at various frequencies (1, 10, 100, and 1,000 Hz) according to an embodiment of the present invention.

With reference to FIG. 25, it may be observed that even when the EGaIn/SHP composite is deformed at a constant strain rate, the impedance value remains relatively constant without significant variations.

FIG. 26 is a graph illustrating a cyclic voltammetry of an EGaIn/SHP composite with (dashed line) and without (solid line) an Alg-CA coating layer according to an embodiment of the present invention.

With reference to FIG. 26, it may be observed that the EGaIn/SHP composite alone exhibits almost negligible charge capacity as measured by cyclic voltammetry, whereas the presence of an Alg-CA coating layer significantly increases the charge capacity values. Therefore, it may be inferred that the device with an Alg-CA coating layer is much more suitable as an electrical stimulation device.

FIG. 27 is photographs illustrating adhesive strengths of a substrate according to a comparative manufacturing example 1-1 and a conductive composite of according to embodiment 1 of the present invention.

With reference to FIG. 27, it may be observed that there is excellent self-bonding between the SHP solid film substrate and the composite made by mixing SHP and EGaIn, achieved through self-bonding. It may also be observed that when using a PDMS film substrate instead of an SHP film substrate, the bonding strength with the EGaIn/SHP composite is not favorable. Therefore, this advantageously facilitates the easy fabrication of devices by allowing self-bonding between the SHP-based EGaIn composite and the SHP film substrate.

FIG. 28 is a graph illustrating an in vitro cell viability of an EGaIn composite on E-SHN with an Alg-CA coating layer (SAFIE device) according to an embodiment of the present invention.

Specifically, E-SHN and the EGaIn composites alone were used as control samples.

With reference to FIG. 28, it may be observed that both the E-SHN and EGaIn composites, as well as the device (SAFIE) created by combining them, show a majority of viable cells in the cell tests, indicating excellent biocompatibility.

FIG. 29 is a graph illustrating the IR spectra of EGaIn with Alg-CA coating layer (dashed line) and only Alg-CA (solid line) according to an embodiment of the present invention.

With reference to FIG. 29, it may be observed that by comparing the IR spectra of the EGaIn/SHP composite with and without an Alg-CA coating layer, changes in the bonding of carbon (C) and oxygen (O) may be confirmed through chemical bond analysis.

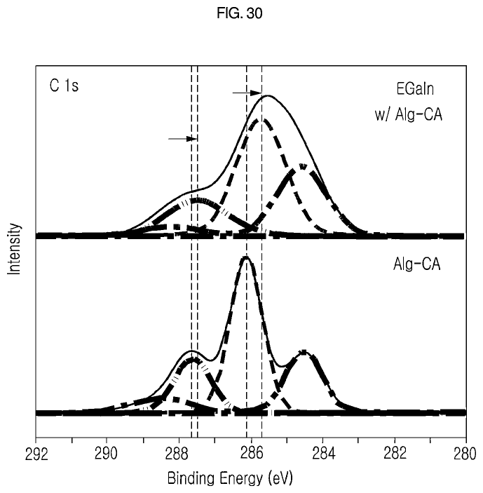

FIG. 30 is a graph illustrating the high-resolution XPS spectra of C 1s for an EGaIn/SHP composite with and without an Alg-CA coating layer according to an embodiment of the present invention.

With reference to FIG. 30, it may be observed that by comparing the binding energy values of C 1s between the Alg-CA standalone sample and the Alg-CA coated EGaIn, changes in the binding energy of C 1s may be confirmed, indicating the occurrence of chemical interactions between Alg-CA and EGaIn.

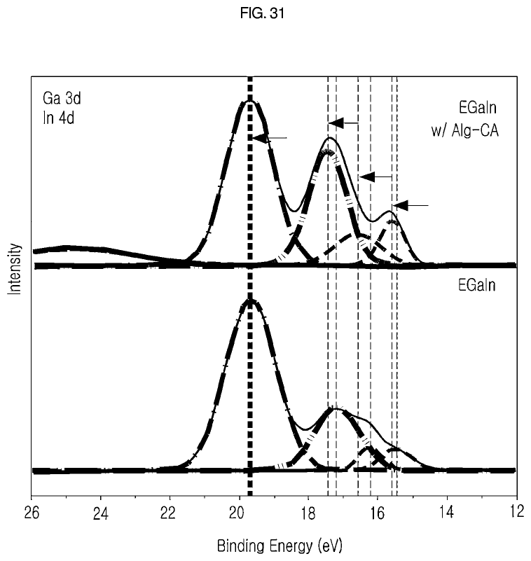

FIG. 31 is a graph illustrating the high-resolution XPS spectra of a Ga 3d and In 4d of the EGaIn/SHP composite with and without an Alg-CA coating layer according to an embodiment of the present invention.

With reference to FIG. 31, it may be observed that the comparison of Ga3d and In 4d binding energies between EGaIn standalone and EGaIn with Alg-CA coating indicates a change in the binding energy values of Ga and In due to the chemical interaction between Alg-CA and Ga ions, as well as between In ions.

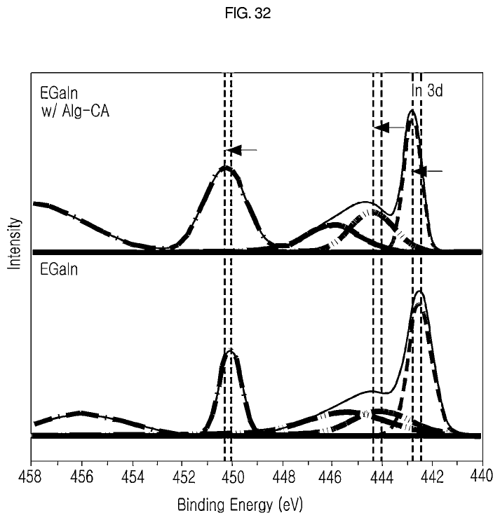

FIG. 32 is a graph illustrating the high-resolution XPS spectra of In 3d for an EGaIn/SHP composite with and without an Alg-CA coating layer according to an embodiment of the present invention.

With reference to FIG. 32, similar to FIG. 21, it may be observed that the comparison of the binding energies of In 3d between EGaIn alone and EGaIn sample with Alg-CA coating, indicating a change in the binding energy of In due to the chemical interaction between Alg-CA and In ions.

FIG. 33 is a graph illustrating the electrocardiograms (ECG) of rat heart stimulation using an EGaIn composite with Alg-CA (top) and without Alg-CA (bottom) according to an embodiment of the present invention. Specifically, in the absence of Alg-CA, the EGaIn composite lost charge injection characteristics, and after several stimulations, the stimulation artifact (gray asterisk) and heart rhythm (arrow) were completely separated. The EGaIn composite with Alg-CA demonstrated stable myocardial capture (arrow) following the stimulation artifact.

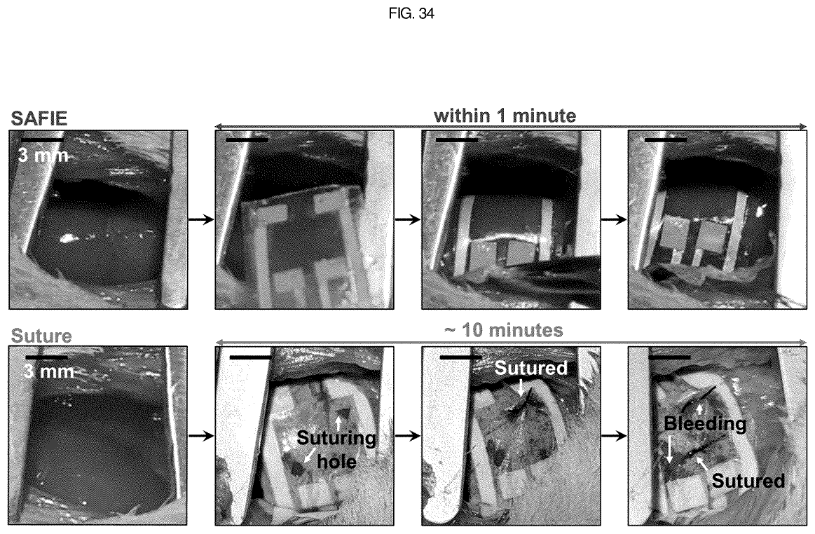

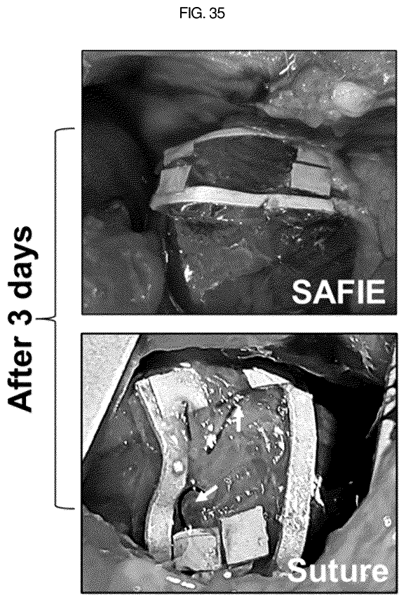

FIG. 34 is photographs illustrating implantation processes of a SAFIE device on a rat heart (top images) and a surgical suturing of the SAFIE device without Alg-CA (bottom images) over time according to an embodiment of the present invention.

With reference to FIG. 34, it may be observed that when the SAFIE device is attached, there is no bleeding, whereas when the SAFIE device is attached without Alg-CA, bleeding is present.

FIG. 35 is photographs illustrating the SAFIE device (top) and the device sutured onto the heart of a rat after three days of epicardial device implantation (bottom) according to an embodiment of the present invention.

With reference to FIG. 35, it may be observed that when using the SAFIE device, it remains well adhered without bleeding even after 3 days, whereas when using the sutured device, bleeding is present.

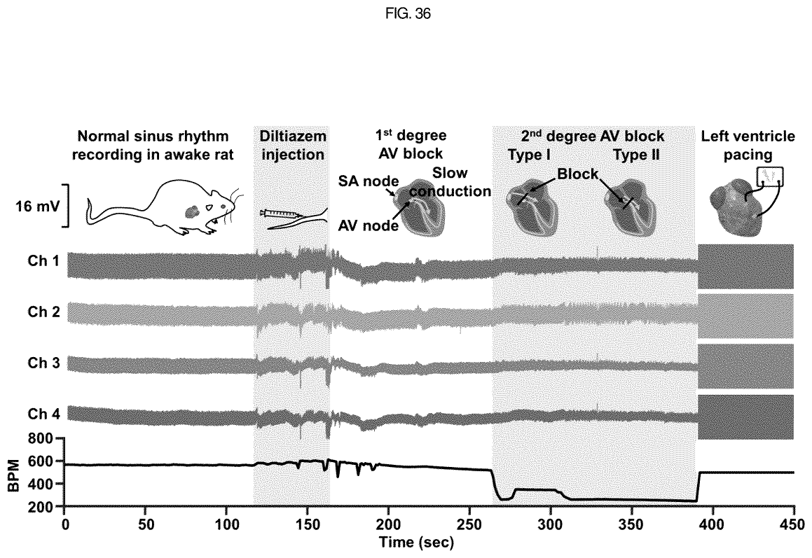

FIG. 36 is a photograph illustrating real-time ECG signals and bpm information received from four detection channels of the SAFIE device before and after drug injection in an induced heart disease model according to an embodiment of the present invention.

A device for measuring for measuring biosignals and electrical stimulation according to an embodiment of the present invention is advantageous in terms of achievement of low mechanical properties, excellent stress relief properties, and sustainable conductivity by incorporating a conductive composite including self-healing polymers and liquid metals.

It should be understood that the advantages of the present invention are not limited to the aforesaid but include all advantages that can be inferred from the detailed description of the present invention or the configuration specified in the claims.

The above description of the present invention is for illustrative purposes only, and it will be understood by those skilled in the art that various modifications and changes may be made thereto without departing from the spirit and scope of the invention. Therefore, it should be understood that the embodiments described above are exemplary and not limited in all respects. For example, each component described as a single type may be implemented in a distributed manner, and similarly, components described as distributed may be implemented in a combined form.

The scope of the invention should be determined by the appended claims, and all changes or modifications derived from the meaning and scope of the claims and equivalent concepts thereof should be construed as being included in the scope of the present invention.

What is claimed is:

1. A device for measuring biosignals and electrical stimulation, the device comprising a conductive composite comprising a self-healing polymer and liquid metal.

2. The device of claim 1, wherein the liquid metal is a eutectic alloy.

3. The device of claim 1, wherein the liquid metal comprises one or more selected from the group consisting of eutectic gallium-indium alloy, eutectic gallium-tin alloy, eutectic gallium-indium-tin alloy, and gallium.

4. The device of claim 1, wherein the liquid metal is 82 to 88 wt % in content based on a total weight of the conductive composite as 100 wt %.

5. The device of claim 1, wherein the liquid metal is dispersed within the conductive composite.

6. The device of claim 1, wherein the conductive composite is arranged on a network fiber layer comprising a fiber of the self-healing polymer.

7. The device of claim 4, wherein the conductive composite is coupled to the network fiber layer.

8. The device of claim 5, further comprising a hydrogel coating layer formed by coating hydrogel on the network fiber layer and the conductive composite.

9. The device of claim 8, wherein the hydrogel is infiltrated into the fiber layer.

10. The device of claim 6, wherein the network fiber layer is porous.

11. The device of claim 6, wherein the network fiber layer comprises a fiber with a diameter of 4.5 to 6.5 μm and a pore with a diameter of 50 to 70 μm.

12. The device of claim 6, wherein the hydrogel penetrate less than 60% of the thickness of the network fiber layer from a contact surface with the network fiber layer.

13. The device of claim 6, wherein the hydrogel is a catechol conjugated polymer.

14. The device of claim 6, wherein the hydrogel undergoes hydrogen bonding or hydrophobic interactions with tissue.

15. The device of claim 1, wherein the self-healing polymer comprises a polymer main chain, a first structural unit containing —HN—C(═O)—NH— capable of forming a strong hydrogen bond, and a second structural unit containing —HN—C(═O)—NH— capable of forming a weak hydrogen bond.

16. The device of claim 1, wherein the polymer main chain comprises at least one selected from polysiloxane and polydialkylsiloxane (where alkyl is C1 to C6), such as polydimethylsiloxane, polyethylene oxide (PEO), polypropylene oxide (PPO), polybutylene oxide (PBO), perfluoropolyether (PFPE), polyolefin, poly(ethylene-co-1-butylene), polybutadiene, hydrogenated polybutadiene, poly(ethylene oxide)-poly(propylene oxide) copolymer, poly(hydroxyalkanoate), styrene-butadiene copolymer (SB), styrene-butadiene-styrene copolymer (SBS), styrene-ethylene-butylene-styrene copolymer (SEBS), ethylene propylene diene rubber (EPDR), acrylic rubber, polychloroprene rubber, polyurethane, fluoro-rubber, butyl rubber, or silicone rubber.

17. The device of claim 15, wherein the first structural unit is represented by Formula 1: 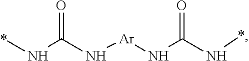
where Ar is a substituted or unsubstituted arylene group of C6 to C30 or a planar heteroarylene group of C3 to C30.

18. The device of claim 15, wherein the second structural unit is represented by Formula 2-1: 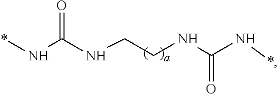
where a is an integer from 5 to 20.

19. The device of claim 15, wherein the second structural unit is represented by Formula 2-2: 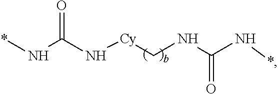
where Cy is a substituted or unsubstituted cyclic alkylene group of C5 to C30, and b is an integer of 1 or 3.

20. The device of claim 1, wherein the biosignal is a biosignal of human or animal tissue.

21. The device of claim 20, wherein the biosignal is a biosignal of cardiac tissue of human or animal.

22. A method for manufacturing a device for measuring biosignals and electrical stimulation, the method comprising: fabricating a network fiber layer by electrospinning self-healing polymer; arranging a conductive composite comprising the self-healing polymer and liquid metal on the network fiber layer; and forming a hydrogel coating layer coated with hydrogel on the network fiber layer and the conductive composite.

23. The method of claim 22, wherein the biosignal is a biosignal of human or animal tissue.

24. The method of claim 22, wherein the biosignal is a biosignal of cardiac tissue of human or animal.

25. A device for drug delivery or implantable in a body, the device comprising the device for measuring a biosignal of electrical stimulation, which is manufactured by the method of claim 22.
